(19) United States
(12) Patent Application Publication
(10) Pub. No.: US 2024/0368260 A1
(43) Pub. Date: Nov. 7, 2024

(54) HUMANIZED ANTI-HUMAN ßIG-H3 PROTEIN AND USES THEREOF

(71) Applicants: INSTITUT NATIONAL DE LA SANTE ET DE LA RECHERCHE MEDICALE, Paris (FR); CENTRE NATIONAL DE LA RECHERCHE SCIENTIFIQUE, Paris (FR); CENTRE LEON BERARD, Lyon (FR); UNIVERSITÉ CLAUDE BERNARD LYON 1, Villeurbanne (FR); KIST (KOREA INSTITUTE OF SCIENCE AND TECHNOLOGY), Seoul (KR)

(72) Inventor: Ana HENNINO, Lyon (FR)

(21) Appl. No.: 18/291,652

(22) PCT Filed: Jul. 28, 2022
(86) PCT No.: PCT/EP2022/071275

§ 371 Date: Jan. 24, 2024

(30) Foreign Application Priority Data
Jul. 29, 2021 (EP) 21306058.5

(51) Int. Cl. C07K 16/22; A61K 39/00; A61P 35/00

(52) U.S. Cl. CPC C07K 16/22; A61P 35/00; A61K 2039/505; C07K 2317/24; C07K 2317/92; C07K 2317/94

(57) ABSTRACT

The tumoral stroma evolution during cancer is playing a key role as it may acts as a physical barrier limiting access of the immune cells to the tumor. Thus, βig-h3 (TGFβi) overexpression in the stroma is of bad prognosis in Pancreatic Ductal Adenocarcinoma and in other cancers. A monoclonal antibody against βig-h3 protein called 18B3 was shown playing a role in directly modulating the anti-tumoral immune response by blocking inhibiting CD8+ T cell activation. From 18B3 the inventors developed humanized antibodies having unexpectedly high affinity, slow dissociation rate and strong thermal stability making them powerful candidates for treating cancers wherein the stroma expresses βig-h3 in vivo. The invention thus relates to these humanized monoclonal antibodies and methods of treatment of such cancers.

### FIELD OF THE INVENTION

The present invention relates to the field of antibodies. In particular, it provides humanized antibodies having specificity to Human βig-h3 protein and uses thereof. Also provided are medical uses, in particular for the treatment of cancers in which the stromal protein βig-h3 is expressed in vivo such as Pancreatic Ductal Adenocarcinoma (PDAC), lung cancer, head and neck cancer, colon cancer, bladder cancer, melanoma, and others as mentioned hereinafter.

### BACKGROUND OF THE INVENTION

The tumoral stroma evolution during cancer is playing a key role as it may acts as a physical barrier limiting access of the immune cells to the tumor. Thus, identifying key molecules expressed or overexpressed in the tumoral stroma and involved in immunosuppression would lead to new therapeutic opportunities. Amongst the stromal proteins, βig-h3 (also known as TGFβi) has been shown that its overexpression in the stroma is of bad prognosis in Pancreatic Ductal Adenocarcinoma (PDAC) and in other cancers (such as, lung cancer, head and neck cancer, colon cancer, bladder cancer, melanoma).

Exploring the potential role of the βig-h3 stromal protein in using the PDAC model (both in mice and humans), it has been determined that this protein decreases the cytotoxic activity of T lymphocytes and helps stiffen the microenvironment, making the tumor less accessible to the immune system.

In mice and humans, the βig-h3 protein is not expressed in the pancreas exocrine compartment in the healthy individual but appears very early in the tumor stroma.

It has thus been proposed modulating tumoral stroma, by targeting some of its key components, such as βig-h3, with specific drugs, to help in therapeutic treatment of solid cancers. It has been proposed that specific depletion of that protein could restore the CD8+ T cell activity and decrease the rigidity of the stroma, thus restoring the access to the tumor.

One standard approach for depleting a protein is to set-up a monoclonal antibody (mAb) specifically addressing key epitope and thus blocking the functional activity of that protein and, subsequently, the in-vivo elimination of the Protein/Antibody complex.

Antibody against βig-h3 protein was shown playing a role in directly modulating the anti-tumoral immune response by blocking inhibiting CD8+ T cell activation (WO 2017/158043). A murine monoclonal antibody directed against the human βig-h3 protein, called 18B3, was described in WO 2020/079164.

### SUMMARY OF THE INVENTION

There is a continuing need for novel, and preferably improved, means for the treatment of cancer. It is thus an object of the present invention to provide improved means for the treatment of cancer. In particular, these improved means are intended to specific depletion of βig-h3 protein, restoration of the CD8+ T cell activity and decrease of the rigidity of the stroma, thus restoring or facilitating the immune system's accessibility to the tumor, ultimately leading to significant tumor reduction and survival rate. It is also intended facilitating medicament's access to the tumor. The present approach for depleting the βig-h3 protein is to set-up humanized monoclonal antibodies (mAb) specifically addressing a key epitope and thus blocking the functional activity of that protein. Subsequently, the in vivo elimination of the Protein/Antibody complex is obtained.

The present invention thus relates to humanized (Hz) antibodies having specificity to βig-h3 protein and uses thereof. These Hz antibodies are βig-h3 antagonists. In particular, the present invention is defined by the claims. These antibodies are humanized versions of the 18B3 antibody. These humanized antibodies have particularly attractive and unexpected properties (e.g. affinity, dissociation rate, thermal stability, productivity in cell culture) and are promising antibodies for therapeutic use and in particular in restoring of the CD8+ T cell activity and decreasing the rigidity of the stroma, thus restoring or facilitating the immune system's and/or medicament' accessibility to the tumor. Thus, these humanized mAbs proved to be successful in in vitro and in vivo functional bioassays.

The mAbs are targeting a region of the βig-h3 protein which is known to be involved in the binding on the integrins (involved in T cells activation pathway) and collagen (involved into modulating the tumoral microenvironment or stroma). The region is the avb3 (αVβ3) integrin-interacting motif, which is present within a fragment corresponding to amino acids (AA) 548-614. Epitope mapping studies showed that the antibody targets the FAS1 4^{th} domain of the βig-h3 protein (linear epitope ALPPRERSRL, SEQ ID NO: 16, which can even be reduced to the eight central amino acids, SEQ ID NO: 30) of the βig-h3-protein (AA residues 549-558). Several affinity and functional bioassays reported herein allows confirming the specific binding of the humanized (Hz) monoclonal antibodies disclosed herein.

In an aspect, the invention concerns humanized anti-βig-h3 monoclonal antibodies or antigen-binding fragments thereof comprising a variable domain VH and a variable domain VL such as the antibody or antigen-binding fragment thereof binds specifically to an epitope of the βig-h3 protein, said epitope being as set forth as sequence SEQ ID NO: 16 or 30, with a high affinity K_{D} of 1 nM or less, preferably of 0.7 nM or less, more preferably of 0.6 or 0.58 nM or less, and a slow dissociation rate K_{d} comprised between 4 and 10 E-04 s⁻¹, preferably between 5 and 8 E-04 s⁻¹, more preferably between 5.5 and 7.5 E-04 s⁻¹, as measured using Surface Plasmon Resonance (SPR). SPR may be measured using a biosensor system such as a Biacore® system.

Affinity and dissociation rate disclosed herein have been measured as described in the part Methods of measure.

In an embodiment, the invention concerns humanized anti-βig-h3 monoclonal antibodies or antigen-binding fragments thereof comprising a variable domain VH and a variable domain VL such as the antibody or antigen-binding fragment thereof binds specifically to an epitope of the βig-h3 protein. Said epitope is as set forth as sequence SEQ ID NO: 16 or 30. The VH domain has sequence as set forth in SEQ ID NO: 4 or 28. SEQ ID NO:28 is a mutated version of SEQ ID NO: 4, i.e. cysteine 102 in H-CDR3 is replaced by serine. The VL is a humanized variant of the murine 18B3 VL domain having a sequence set forth as SEQ ID NO: 18. This combination of VH and VL provides to the humanized anti-βig-h3 monoclonal antibodies or antigen-binding fragments thereof a high and unexpected thermal resistance, i.e. a DSC which is equal or above 80° C., in particular comprised between 80 and 83, 83.2, 83.5 or 84° C., preferably between 81 and about 83 or 83.2° C. DSC is as measured using the method described in the part Methods of measure. In an aspect, this humanized anti-βig-h3 monoclonal antibodies or antigen-binding fragments thereof further binds to said epitope with a high affinity K_{D} of 1 nM or less, preferably of 0.7 nM or less, more preferably of 0.6 or 0.58 nM or less, and/or a slow dissociation rate K_{d} comprised between 4 and 10 E-04 s⁻¹, preferably between 5 and 8 E-04 s⁻¹, more preferably between 5.5 and 7.5 E-04 s⁻¹, as measured using Surface Plasmon Resonance (SPR). SPR may be measured using a biosensor system such as a Biacore® system.

The humanized antibodies of the invention may allow depleting the βig-h3 protein.

The humanized antibodies of the invention may restore the CD8+ T cell activity and/or decrease the rigidity of the stroma, thus restoring the access to the tumor. These antibodies may thus be used in combination with another anti-tumor agent(s) that may access more easily to the tumor owing the effect of the anti-βig-h3 antibodies on the stroma.

The present invention also relates to pharmaceutical compositions comprising at least one humanized monoclonal antibody or antigen-binding fragment thereof, and a pharmaceutically acceptable vehicle.

The present invention also relates to pharmaceutical compositions, pharmaceutical combinations or kits of part comprising at least one humanized monoclonal antibody or antigen-binding fragment thereof, and another anti-tumoral medicament, such as an antibody, in particular a monoclonal antibody or fragment thereof.

The present invention also relates to such antibodies, pharmaceutical compositions, pharmaceutical combinations or kits of part, for use in the prevention or treatment of cancer, in depleting the βig-h3 protein, in restoring or activating the CD8+ T cell activity and/or in decreasing the rigidity of the stroma and favoring the access to the tumor to other anti-tumoral medicaments, such as antibodies, monoclonal antibodies.

The present invention also relates to methods of prevention or treatment of cancer, comprising administering to a patient in need thereof of an effective amount of such antibodies, pharmaceutical compositions, pharmaceutical combinations or kits of part. The present invention also relates to depleting the βig-h3 protein, restoring or activating the CD8+ T cell activity and/or in decreasing the rigidity of the stroma and favoring the access to the tumor to other anti-tumoral medicaments, such as antibodies, monoclonal antibodies.

### DETAILED DESCRIPTION OF THE INVENTION

### Humanized Antibodies

In an aspect, the invention concerns humanized anti-βig-h3 monoclonal antibodies or antigen-binding fragments thereof comprising a variable domain VH and a variable domain VL such as the antibody or antigen-binding fragment thereof binds specifically to an epitope of the βig-h3 protein. The epitope is preferably as set forth as sequence SEQ ID NO: 16 or 30. Of course, it cannot be excluded the antibodies of the invention or their fragments are capable of binding to a βig-h3 protein fragment bigger than SEQ ID NO: 16 or 30 and comprising this sequence. The binding of the antibodies or fragments thereof may occur with a surprising high level of affinity, in particular with a high affinity K_{D} of 1 nM or less, preferably of 0.7 nM or less, more preferably of 0.6 or 0.58 nM or less, as measured using Surface Plasmon Resonance (SPR). Remarkably and unexpectedly, the Hz antibodies or their fragments present after binding a slow dissociation rate K_{d}. This slow dissociation rate may be comprised between 4 and 10 E-04 s⁻¹, preferably between 5 and 8 E-04 s⁻¹, more preferably between 5.5 and 7.5 E-04 s⁻¹, as measured using SPR. SPR may be measured using a biosensor system such as a Biacore® system.

The sequences of interest in the present application are indicated in the following

TABLE 1

| | | |
|---|---|---|
| Mab | | SEQ ID |
| domain | Sequence | NO: |

H-330 variant (Based on Kabat methodology)

| | | |
|---|---|---|
| H-CDR1 | DYYMY | 1 |
| H-CDR2 | TISDGGIYKYYADSVKG | 2 |
| H-CDR3 | GWDRYDSWFAC | 3 |
| VH | QVQLVESGGGVVQPGGSLRLSCAASGFTFSDYYMYWVRQ | 4 |
| | APGKGLEWVATISDGGIYKYYADSVKGRFTISRDSSKNTLY | |
| | LQMNSLRAEDTAVYYCVRGWDRYDSWFACWGQGTTVTVSS | |

H-311 variant (Based on Kabat methodology)

| | | |
|---|---|---|
| H-CDR1 | DYYMY | 1 |
| H-CDR2 | TISDGGIYIYYADSVKG | 5 |
| H-CDR3 | GWDRYDSWFAC | 3 |
| VH | QVQLVESGGGLVKPGGSLRLSCAASGFTFSDYYMYWIRQ | 6 |
| | APGKGLEWVATISDGGIYIYYADSVKGRFTISRDSAKNSLY | |
| | LQMNSLRAEDTAVYYCVRGWDRYDSWFACWGQGTTVTVSS | |

L-41 variant (Based on Kabat methodology)

| | | |
|---|---|---|
| L-CDR1 | KSSQSLLYSSNQKNYLA | 7 |
| L-CDR2 | WASTRES | 8 |
| L-CDR3 | QQYYRYPYT | 9 |
| VL | DIVMTQSPDSLAVSLGERATMNCKSSQSLLYSSNQKNYLA | 10 |
| | WYQQKPGQPPKLLIYWASTRESGVPDRFSGSGSGTDFTL | |
| | TISSLQAEDVAVYYCQQYYRYPYTFGQGTKLEIK | |

L-228 variant (Based on Kabat methodology)

| | | |
|---|---|---|
| L-CDR1 | RSSQSLLYSSNQKNYLA | 11 |
| L-CDR2 | WGSTRES | 12 |
| L-CDR3 | QQYYRYPYT | 9 |
| VL | DIVMTQSPLSLPVTPGEPASMSCRSSQSLLYSSNQKNYLA | 13 |
| | WYLQKPGQSPQLLIYWGSTRESGVPDRFSGSGSGTDFTL | |
| | KISRVEAEDVGVYYCQQYYRYPYTFGQGTKLEIK | |

Constant domain human for Heavy chain Heavy Human IgG1 m1, 17: CH1-CH2-CH3

| | |
|---|---|
| ASTKGPSVFPLAPSSKSTSGGTAALGCLVKDYFPEPVTVSWNS | 14 |
| GALTSGVHTFPAVLQSSGLYSLSSVVTVPSSSLGTQTYICNVNH | |
| KPSNTKVDKKVEPKSCDKTHTCPPCPAPELLGGPSVFLFPPKP | |
| KDTLMISRTPEVTCVVVDVSHEDPEVKFNWYVDGVEVHNAKT | |
| KPREEQYNSTYRVVSVLTVLHQDWLNGKEYKCKVSNKALPAP | |
| IEKTISKAKGQPREPQVYTLPPSRDELTKNQVSLTCLVKGFYP | |
| SDIAVEWESNGQPENNYKTTPPVLDSDGSFFLYSKLTVDKSR | |
| WQQGNVFSCSVMHEALHNHYTQKSLSLSPGK | |

CL-Constant domain human for Light chain (Kappa)-Light Human Km3

| | |
|---|---|
| RTVAAPSVFIFPPSDEQLKSGTASVVCLLNNFYPREAKVQWK | 15 |
| VDNALQSGNSQESVTEQDSKDSTYSLSSTLTLSKADYEKHK | |
| VYACEVTHQGLSSPVTKSFNRGEC | |

Epitope on Big-h3-protein (AA residues 549-558)

| | | |
|---|---|---|
| Epitope | ALPPRERSRL | 16 |

Sequences of murine 18B3 antibody

| | | |
|---|---|---|
| VH | EVQLVESGGGLVKPGGSLKLSCAASGFTFSDYYMYWVRQTPEK | 17 |
| | RLEWVATISDGGIYTYYPDSVKGRFTISRDSAKNNLYLQMTSLKS | |
| | DDTAMYYCVRGWDRYDSWFACWGQGTLVTVSA | |
| VL | DIVMSQSPSSLVVSAGEKVTMTCKSSQSLLYSSNQKNYLAWYR | 18 |
| | QKPGQSPKLLIYWASTRESGVPDRFTGSGSGTDFTLTISSVKAED | |
| | LAVYYCQQYYRYPYTFGGGTKLEIK | |
| L-CDR1 | KSSQSLLYSSNQKNYLA | 7 |
| L-CDR2 | WASTRES | 8 |
| L-CDR3 | QQYYRYPYT | 9 |

Nucleic acid sequences

| | | |
|---|---|---|
| 18B3 VH | gaagtgcagctggtggaaagcggcggcggcctggtgaaaccgggcggcagcctgaaac | 19 |
| | tgagctgcgcggcgagcggctttacctttagcgattattatatgtattgggtgcgccagacccc | |
| | ggaaaaacgcctggaatgggtggcgaccattagcgatggcggcatttatacctattatccgg | |
| | atagcgtgaaaggccgctttaccattagccgcgatagcgcgaaaaacaacctgtatctgca | |
| | gatgaccagcctgaaaagcgatgataccgcgatgtattattgcgtgcgcggctgggatcgct | |
| | atgatagctggtttgcgtgctggggccagggcaccctggtgaccgtgagcgcg | |
| 18B3 VL | gatattgtgatgagccagagcccgagcagcctggtggtgagcgcgggcgaaaaagtgac | 20 |
| | catgacctgcaaaagcagccagagcctgctgtatagcagcaaccagaaaaactatctggc | |
| | gtggtatcgccagaaaccgggccagagcccgaaactgctgatttattgggcgagcacccg | |
| | cgaaagcggcgtgccggatcgctttaccggcagcggcagcggcaccgattttaccctgac | |
| | cattagcagcgtgaaagcggaagatctggcggtgtattattgccagcagtattatcgctatcc | |
| | gtatacctttggcggcggcaccaaactggaaattaaa | |
| H-330 | caggtgcagctggtggaaagcggcggcggcgtggtgcagccgggcggcagcctgcgcct | 21 |
| | gagctgcgcggcgagcggctttacctttagcgattattatatgtattgggtgcgccaggcgcc | |
| | gggcaaaggcctggaatgggtggcgaccattagcgatggcggcatttataaatattatgcg | |
| | gatagcgtgaaaggccgctttaccattagccgcgatagcagcaaaaacaccctgtatctgc | |
| | agatgaacagcctgcgcgcggaagataccgcggtgtattattgcgtgcgcggctgggatcg | |
| | ctatgatagctggtttgcgtgctggggccagggcaccaccgtgaccgtgagcagc | |
| H-311 | caggtgcagctggtggaaagcggcggcggcctggtgaaaccgggcggcagcctgcgcct | 22 |
| | gagctgcgcggcgagcggctttacctttagcgattattatatgtattggattcgccaggcgccg | |
| | ggcaaaggcctggaatgggtggcgaccattagcgatggcggcatttatatttattatgoggat | |
| | agcgtgaaaggccgctttaccattagccgcgatagcgcgaaaaacagcctgtatctgcag | |
| | atgaacagcctgcgcgcggaagataccgcggtgtattattgcgtgcgcggctgggatcgct | |
| | atgatagctggtttgcgtgctggggccagggcaccaccgtgaccgtgagcagc | |
| L-41 | gatattgtgatgacccagagcccggatagcctggcggtgagcctgggcgaacgcgcgacc | 23 |
| | atgaactgcaaaagcagccagagcctgctgtatagcagcaaccagaaaaactatctggc | |
| | gtggtatcagcagaaaccgggccagccgccgaaactgctgatttattgggcgagcacccg | |
| | cgaaagcggcgtgccggatcgctttagcggcagcggcagcggcaccgattttaccctgac | |
| | cattagcagcctgcaggcggaagatgtggcggtgtattattgccagcagtattatcgctatcc | |
| | gtatacctttggccagggcaccaaactggaaattaaa | |
| L-228 | gatattgtgatgacccagagcccgctgagcctgccggtgaccccgggcgaaccggcgag | 24 |
| | catgagctgccgcagcagccagagcctgctgtatagcagcaaccagaaaaactatctggc | |
| | gtggtatctgcagaaaccgggccagagcccgcagctgctgatttattggggcagcacccgc | |
| | gaaagcggcgtgccggatcgctttagcggcagcggcagcggcaccgattttaccctgaaa | |
| | attagccgcgtggaagcggaagatgtgggcgtgtattattgccagcagtattatcgctatccgt | |
| | atacctttggccagggcaccaaactggaaattaaa | |
| Human | GCCAGCACCAAGGGCCCCAGCGTGTTCCCCCTGGCCCCCAG | 25 |
| IgG1m1, | CAGCAAGAGCACCAGCGGCGGCACCGCCGCCCTGGGCTGCC | |
| 17 | TGGTGAAGGACTACTTCCCCGAGCCCGTGACCGTGAGCTGGA | |
| | ACAGCGGCGCCCTGACCAGCGGCGTGCACACCTTCCCCGCC | |
| | GTGCTGCAGAGCAGCGGCCTGTACAGCCTGAGCAGCGTGGT | |
| | GACCGTGCCCAGCAGCAGCCTGGGCACCCAGACCTACATCTG | |
| | CAACGTGAACCACAAGCCCAGCAACACCAAGGTGGACAAGAA | |
| | GGTGGAGCCCAAGAGCTGCGACAAGACCCACACCTGCCCCC | |
| | CCTGCCCCGCCCCCGAGCTGCTGGGCGGCCCCAGCGTGTTC | |
| | CTGTTCCCCCCCAAGCCCAAGGACACCCTGATGATCAGCAGG | |
| | ACCCCCGAGGTGACCTGCGTGGTGGTGGACGTGAGCCACGA | |
| | GGACCCCGAGGTGAAGTTCAACTGGTACGTGGACGGCGTGG | |
| | AGGTGCACAACGCCAAGACCAAGCCCAGGGAGGAGCAGTAC | |
| | AACAGCACCTACAGGGTGGTGAGCGTGCTGACCGTGCTGCAC | |
| | CAGGACTGGCTGAACGGCAAGGAGTACAAGTGCAAGGTGAG | |
| | CAACAAGGCCCTGCCCGCCCCCATCGAGAAGACCATCAGCAA | |
| | GGCCAAGGGCCAGCCCAGGGAGCCCCAGGTGTACACCCTGC | |
| | CCCCCAGCAGGGACGAGCTGACCAAGAACCAGGTGAGCCTG | |
| | ACCTGCCTGGTGAAGGGCTTCTACCCCAGCGACATCGCCGTG | |
| | GAGTGGGAGAGCAACGGCCAGCCCGAGAACAACTACAAGAC | |
| | CACCCCCCCCGTGCTGGACAGCGACGGCAGCTTCTTCCTGTA | |
| | CAGCAAGCTGACCGTGGACAAGAGCAGGTGGCAGCAGGGCA | |
| | ACGTGTTCAGCTGCAGCGTGATGCACGAGGCCCTGCACAACC | |
| | ACTACACCCAGAAGAGCCTGAGCCTGAGCCCCGGCAAG | |
| Human | AGGACCGTGGCCGCCCCCAGCGTGTTCATCTTCCCCCCCAGC | 26 |
| Km3 | GACGAGCAGCTGAAGAGCGGCACCGCCAGCGTGGTGTGCCT | |
| | GCTGAACAACTTCTACCCCAGGGAGGCCAAGGTGCAGTGGAA | |
| | GGTGGACAACGCCCTGCAGAGCGGCAACAGCCAGGAGAGCG | |
| | TGACCGAGCAGGACAGCAAGGACAGCACCTACAGCCTGAGCA | |
| | GCACCCTGACCCTGAGCAAGGCCGACTACGAGAAGCACAAG | |
| | GTGTACGCCTGCGAGGTGACCCACCAGGGCCTGAGCAGCCC | |
| | CGTGACCAAGAGCTTCAACAGGGGCGAGTGC | |
| H′-CDR3 | GWDRYDSWFAS | 27 |
| VH-1 | QVQLVESGGGVVQPGGSLRLSCAASGFTFSDYYMYWVRQ | 28 |
| | APGKGLEWVATISDGGIYKYYADSVKGRFTISRDSSKNTLY | |
| | LQMNSLRAEDTAVYYCVRGWDRYDSWFASWGQGTTVTVSS | |
| H330C102S | caggtgcagctggtggaaagcggcggcggcgtggtgcagccgggcggcagcctgcgcct | 29 |
| | gagctgcgcggcgagcggctttacctttagcgattattatatgtattgggtgcgccaggcgcc | |
| | gggcaaaggcctggaatgggtggcgaccattagcgatggcggcatttataaatattatgcg | |
| | gatagcgtgaaaggccgctttaccattagccgcgatagcagcaaaaacaccctgtatctgc | |
| | agatgaacagcctgcgcgcggaagataccgcggtgtattattgcgtgcgcggctgggatcg | |
| | ctatgatagctggtttgcgagctggggccagggcaccaccgtgaccgtgagcagc | |
| Epitope | LPPRERSR | 30 |
| L-315 | EIVMTQSPATLSVSPGERATMSCRASQSLLYSSNQKNYLAWYQ | 31 |
| | QKPGQAPRLLIYWASTRETGIPARFSGSGSGTEFTLTISSLQSED | |
| | FAVYYCQQYYRYPYTFGQGTKLEIK | |
| H-169 | QVQLVQSGAEVKKPGSSVKVSCKASGFTFSDYYMYWVRQAPG | 32 |
| | QGLEWVGTISDGGIYAYYAQKVQGRFTITRDSSTSTLYLELSSLR | |
| | SEDTAVYYCVRGWDRYDSWFACWGQGTTVTVSS | |

Legend:
in VL and VH, the CDRs according to Kabat are in bold, the other amino acids are in the FRs, specific amino acids differing from the murine are sub-lined.

Two humanized VH domains (H-330 and H-311) and two humanized VL domains (L-41 and L-228) were created, and are each an object of the invention, as are their VH/VL combinations in a monoclonal antibody or fragment thereof. The humanized VH domain H-169 and the humanized VL domain L-315 are also objects of the present invention, as well as their combination with a VL or VH domain of the invention in a monoclonal antibody or fragment thereof.

A mutated version of H-330 has also been created, with the mutation of cysteine 102 into serine. This mutated version is called H330 V1.2 (whereas original H330 is V1) or H330C102S, and has sequence as set forth in SEQ ID NO: 28. This mutation occurs in the H-CDR-3, and the mutated H-CDR3 has sequence as set forth in SEQ ID NO: 27.

These VH domains, including the mutated one, can be combined in order to design antibody binding domains H-330/L-41, H-330/L-228, H-330 V1.2/L-41, H-330 V1.2/L-228, H-311/L-41, and H-311/L-228, each one being an object of the invention. The humanized VH domains H-330, H-330 V1.2 and H-311, especially the domains H-330 and H-330 V1.2, may also be combined with any humanized VL domain derived from m18B3 monoclonal antibody. In particular, the humanized VL domain comprises the L-CDR1, L-CDR2 and L-CDR3 of sequence as set forth at SEQ ID NO: 7, 8, respectively 9; or the L-CDR1, L-CDR2 and L-CDR3 of sequence as set forth at SEQ ID NO: 11, 12, respectively 9. These combination are deemed specifically binding to the βig-h3 protein, and more particularly the FAS1 4^{th} domain of the βig-h3 protein (epitope as set forth as SEQ ID NO: 16 or 30) of the βig-h3-protein (AA residues 549-558). Binding with a good affinity, or with a very high affinity as described above, may be verified using the methods described herein, especially the SPR method, e.g. using a biosensor system such as a Biacore® system, in order to qualify a candidate comprising domain H-330, H-330 V1.2 or H-311 and a humanized domain derived from m18B3. The dissociation rate may be tested with the same method to qualify those candidates.

In terms of affinity measured by ELISA, the humanized variants are not statistically significantly different as compared to the chimeric 18B3, showing that the humanization process did not altered the affinity measured by ELISA. Using Biacore® system, all these humanized mAbs have an affinity (KD) in the sub-nanomolar range, and, surprisingly, a slow dissociation rate, slower than the mouse 18B3 and the chimeric 18B3. Humanized variant H-330/L-228 shows the best affinity among the humanized mAbs.

Thus in some aspects, the invention relates to the humanized anti-βig-h3 monoclonal antibodies or antigen-binding fragment thereof, comprising either H-330/L-41, H-330/L-228, H-330 V1.2/L-41, H-330 V1.2/L-228, H-311/L-41, or H-311/L-228. These antibodies or antigen-binding fragment thereof binds specifically to an epitope of the βig-h3 protein, said epitope being as set forth as sequence SEQ ID NO: 16 or 30 (or a longer sequence as described above). This binding occurs with a high affinity K_{D}, in particular of 1 nM or less, preferably of 0.7 nM or less, more preferably of 0.6 or 0.58 nM or less, as measured using SPR. This binding occurs advantageously with a slow dissociation rate K_{d}, in particular comprised between 4 and 10 E-04 s⁻¹, preferably between 5 and 8 E-04 s⁻¹, more preferably between 5.5 and 7.5 E-04 s⁻¹, as measured using SPR. SPR may be measured using a biosensor system such as a Biacore® system.

H-330 and H-330 V1.2, as exemplified in its combination with all the tested L-variants, either L-41, or L-228, provides to the monoclonal antibody an unexpectedly high thermostability as per DSC, which is above 80° C., in particular comprised between 81 and 83, 83.2 or 83.5° C. Additional data presented in the Examples, with other combinations of VH and VL domains show that H-330 is responsible for this elevated thermal stability whatever the complementary VH domain. The thermal stability of H-330 V1.2 having mutation C102S remains high and above 80° C. H-330 and H-330 V1.2, as exemplified in its combination with all the tested L-variants, either L-41, or L-228, also provides to the monoclonal antibody an unexpectedly high productivity in transient expression in CHO cells, which is above 200 μg/ml, in particular comprised between 230 and 300 μg/ml. This is accompanied with very good affinity by ELISA and Biacore®, and the best Biacore® affinity for the humanized variant H-330/L-228. These particular properties linked to the presence of H-330 or its mutated version C102S in the monoclonal antibodies are surprising with respect to the results obtained with H-311 used in combination with the same VL variants, insofar as there are only 6 amino acid differences between H-330 and H-311.

The Heavy Chain H-330, in its V1 version (SEQ ID NO: 4) or its C102S V1.2 mutated version (SEQ ID NO: 28) demonstrate a high and unexpected thermal stability when associated with different Light Chains (L-41, L-228, L-315), in monoclonal antibodies binding to an epitope of the βig-h3 protein (epitope of sequence SEQ ID NO: 16 or 30) as disclosed herein. The variants with the mutation of cysteine to serine in position 102 of the Heavy Chain showed preserved reactivity and stability properties similar to the unmutated mAbs versions (H-330 V1), and represent valuable candidates for druggability purposes.

DSC disclosed herein and transient expression in CHO have been measured as described in the part Methods of measure.

In a particular aspect, the invention relates to a humanized anti-βig-h3 monoclonal antibody or an antigen-binding fragment thereof, wherein the antibody and the antigen-binding fragment specifically binds to the βig-h3 protein, preferably to the epitope as set forth as sequence SEQ ID NO: 16 or 30, especially with the binding affinity and/or dissociation rate as mentioned above (a high affinity K_{D}, in particular of 1 nM or less, preferably of 0.7 nM or less, more preferably of 0.6 or 0.58 nM or less, as measured using SPR; slow dissociation rate K_{d}, in particular comprised between 4 and 10 E-04 s⁻¹, preferably between 5 and 8 E-04 s⁻¹, more preferably between 5.5 and 7.5 E-04 s⁻¹, as measured using SPR), and comprises:
(a) a variable domain VH (comprising the CDRs of H-330 variant or of H-330 V1.2) comprising:
   a H-CDR1 having a sequence set forth as SEQ ID NO: 1;
   a H-CDR2 having a sequence set forth as SEQ ID NO: 2;
   a H-CDR3 having a sequence set forth as SEQ ID NO: 3 or 27;
(b) a variable domain VL which is a humanized variant of the 18B3 monoclonal antibody, say a humanized variant of the 18B3 VL domain having a sequence set forth as SEQ ID NO: 18.

In an embodiment, the antibody comprises the VH domain having the sequence set forth as SEQ ID NO: 4.

This monoclonal antibody or antigen-binding fragment thereof specifically binds to the βig-h3 protein, and more particularly the FAS1 4^{th} domain of the βig-h3 protein (epitope as set forth as SEQ ID NO: 16 or 30). This binding occurs with a high affinity K_{D} of 1 nM or less, preferably of 0.7 nM or less, more preferably of 0.6 or 0.58 nM or less, and a slow dissociation rate K_{d} comprised between 4 and 10 E-04 s⁻¹, preferably between 5 and 8 E-04 s⁻¹, more preferably between 5.5 and 7.5 E-04 s⁻¹, as measured using SPR. SPR may be measured using a biosensor system such as a Biacore® system.

In another particular aspect, the invention relates to a humanized anti-βig-h3 monoclonal antibody or an antigen-binding fragment thereof, wherein the antibody and the antigen-binding fragment specifically binds to the βig-h3 protein, and comprises:
(a) a variable domain VH (comprising the CDRs of H-330 variant or of H-330 V1.2) comprising:
   a H-CDR1 having a sequence set forth as SEQ ID NO: 1;
   a H-CDR2 having a sequence set forth as SEQ ID NO: 2;
   a H-CDR3 having a sequence set forth as SEQ ID NO: 3 or 27;
(b) a variable domain VL (comprising the CDRs of L-41 variant) comprising
   a L-CDR1 having a sequence set forth as SEQ ID NO: 7;
   a L-CDR2 having a sequence set forth as SEQ ID NO: 8;
   a L-CDR3 having a sequence set forth as SEQ ID NO: 9.

In an embodiment, the antibody comprises the VH domain having the sequence set forth as SEQ ID NO: 4 or 28 and/or the VL domain having a sequence set forth as SEQ ID NO: 10.

This monoclonal antibody or antigen binding fragment thereof:
binds to the βig-h3 protein with a K_{D} of about 5.8 E-10 M or less, in particular between about 5 E-10 and about 5.8 E-10 M or less, especially about 5.35 E-10 M; and/or
binds to the βig-h3 protein with a K_{d} of about 6 E-04 s⁻¹ or more, in particular between about 6.2 E-04 and about 7 E-04 s⁻¹, especially about 6.59 E-04 s⁻¹; and/or
has a stability in DSC (Tm Fab) of about 79° C. or more, in particular between about 79 and about 83, 83.2 or 83.5° C., typically about 81.3° C.; and/or
has a good productivity in transient expression in CHO cells, measure was about 275 μg/ml.

In an embodiment, this humanized anti-βig-h3 monoclonal antibody (H-330/L-41 or H-330 V1.2/L-41) or an antigen-binding fragment thereof, comprises:
a VH domain having a sequence set forth as SEQ ID NO: 4 or 28;
a VL domain having a sequence set forth as SEQ ID NO: 10.

In an embodiment, said humanized anti-βig-h3 antibody comprises:
a heavy chain comprising said variable domain and a constant domain CH, such as a CH having a sequence set forth as SEQ ID NO: 14;
a light chain comprising said variable domain and a constant domain CL, such as a CL having a sequence set forth as SEQ ID NO: 15.

In another particular aspect, the invention relates to a humanized anti-βig-h3 monoclonal antibody or an antigen-binding fragment thereof, wherein the antibody and the antigen-binding fragment specifically binds to the βig-h3 protein, and comprises:
(a) a variable domain VH (comprising the CDRs of H-330 variant or of H-330 V1.2) comprising:
   a H-CDR1 having a sequence set forth as SEQ ID NO: 1;
   a H-CDR2 having a sequence set forth as SEQ ID NO: 2;
   a H-CDR3 having a sequence set forth as SEQ ID NO: 3 or 27;
(b) a variable domain VL (comprising the CDRs of L-228 variant) comprising
   a L-CDR1 having a sequence set forth as SEQ ID NO: 11;
   a L-CDR2 having a sequence set forth as SEQ ID NO: 12;
   a L-CDR3 having a sequence set forth as SEQ ID NO: 9.

In an embodiment, the antibody comprises the VH domain having the sequence set forth as SEQ ID NO: 4 or 28 and/or the VL domain having a sequence set forth as SEQ ID NO: 13.

This monoclonal antibody or antigen binding fragment thereof:
binds to the βig-h3 protein with a K_{D} of about 5 E-10 M or less, in particular about 4.5 E-10 M and about 5E-10 M, typically about 4.76 E-10 M; and/or
binds to the βig-h3 protein with a K_{d} of about 5 E-04 s⁻¹ or more, in particular between about 5.5 E-04 and about 6 E-04 s⁻¹, especially about 5.83 E-04 s⁻¹; and/or
has a stability in DSC (Tm Fab) of 78° C. or more, especially about 78 to 82° C., typically about 80.2° C.; and/or
has a good productivity in transient expression in CHO cells, measure was about 249 μg/ml.

In an embodiment, this humanized anti-βig-h3 monoclonal antibody (H-330/L-228 or H-330 V1.2/L-228) or an antigen-binding fragment thereof, comprises:
a VH domain having a sequence set forth as SEQ ID NO: 4 or 28;
a VL domain having a sequence set forth as SEQ ID NO: 13.

In an embodiment, said humanized anti-βig-h3 monoclonal antibody comprises:
a heavy chain comprising said variable domain and a constant domain CH, such as a CH having a sequence set forth as SEQ ID NO: 14;
a light chain comprising said variable domain and a constant domain CL, such as a CL having a sequence set forth as SEQ ID NO: 15.

In another particular aspect, the invention relates to a humanized anti-βig-h3 monoclonal antibody or an antigen-binding fragment thereof, wherein the antibody and the antigen-binding fragment specifically binds to the βig-h3 protein, and comprises:
(a) a variable domain VH (comprising the CDRs of H-311 variant) comprising:
   a H-CDR1 having a sequence set forth as SEQ ID NO: 1;
   a H-CDR2 having a sequence set forth as SEQ ID NO: 5;
   a H-CDR3 having a sequence set forth as SEQ ID NO: 3;
(b) a variable domain VL (comprising the CDRs of L-41 variant) comprising
   a L-CDR1 having a sequence set forth as SEQ ID NO: 7;
   a L-CDR2 having a sequence set forth as SEQ ID NO: 8;
   a L-CDR3 having a sequence set forth as SEQ ID NO: 9.

In an embodiment, the antibody comprises the VH domain having the sequence set forth as SEQ ID NO: 6 and/or the VL domain having a sequence set forth as SEQ ID NO: 10.

This monoclonal antibody or antigen binding fragment thereof:
binds to the βig-h3 protein with a K_{D} of 5 E-10 M or less, in particular between about 4.5 and 5 E-10 M, especially about 4.82 E-10 M; and/or
binds to the βig-h3 protein with a K_{d} of about 6.8 E-04 s⁻¹ or more, in particular between about 7 E-04 and about 7.5 E-04 s⁻¹, especially about 7.26 E-04 s⁻¹; and/or
has a stability in DSC (Tm Fab) of 75° C. or more, in particular of between about 75 and about 79° C., typically about 77° C.

In an embodiment, this humanized anti-βig-h3 monoclonal antibody (H-311/L-41) or an antigen-binding fragment thereof, comprises:
a VH domain having a sequence set forth as SEQ ID NO: 6;
a VL domain having a sequence set forth as SEQ ID NO: 10.

In an embodiment, said humanized anti-βig-h3 antibody comprises:
a heavy chain comprising said variable domain and a constant domain CH, such as a CH having a sequence set forth as SEQ ID NO: 14;
a light chain comprising said variable domain and a constant domain CL, such as a CL having a sequence set forth as SEQ ID NO: 15.

In another particular aspect, the invention relates to a humanized anti-βig-h3 monoclonal antibody or an antigen-binding fragment thereof, wherein the antibody and the antigen-binding fragment specifically binds to the βig-h3 protein, and comprises:
(a) a variable domain VH (comprising the CDRs of H-311 variant) comprising:
   a H-CDR1 having a sequence set forth as SEQ ID NO: 1;
   a H-CDR2 having a sequence set forth as SEQ ID NO: 5;
   a H-CDR3 having a sequence set forth as SEQ ID NO: 3;
(b) a variable domain VL (comprising the CDRs of L-228 variant) comprising
   a L-CDR1 having a sequence set forth as SEQ ID NO: 11;
   a L-CDR2 having a sequence set forth as SEQ ID NO: 12;
   a L-CDR3 having a sequence set forth as SEQ ID NO: 9.

In an embodiment, the antibody comprises the VH domain having the sequence set forth as SEQ ID NO: 6 and/or the VL domain having a sequence set forth as SEQ ID NO: 13.

This monoclonal antibody or antigen binding fragment thereof:
binds to the βig-h3 protein with a K_{D} of 5 E-10 M or less, in particular between about 4.5 and 5 E-10 M, especially about 4.9 E-10 M; and/or
binds to the βig-h3 protein with a K_{d} of about 6.5 E-04 s⁻¹ or more, in particular between about 6.8 E-04 and about 7.3 E-04 s⁻¹, especially about 7.07 E-04 s⁻¹; and/or
has a stability in DSC (Tm Fab) of about 73.5° C. or more, in particular of about 73.5 and about 77.5° C., typically about 75.5° C.

In an embodiment, this humanized anti-βig-h3 monoclonal antibody (H-311/L-228) or an antigen-binding fragment thereof, comprises:
a VH domain having a sequence set forth as SEQ ID NO: 6;
a VL domain having a sequence set forth as SEQ ID NO: 13.

In an embodiment, said humanized anti-βig-h3 monoclonal antibody comprises:
a heavy chain comprising said variable domain and a constant domain CH, such as a CH having a sequence set forth as SEQ ID NO: 14;
a light chain comprising said variable domain and a constant domain CL, such as a CL having a sequence set forth as SEQ ID NO: 15.

In an embodiment, the humanized antibodies as disclosed herein comprise a human IgG1 constant domain, preferably the Constant domain human for Heavy chain Heavy Human IgG1 m1,17 of SEQ ID NO: 14, and/or a constant domain for Light Chain, especially Kappa, preferably a Constant domain human for Light chain (Kappa)-Light Human Km3 of SEQ ID NO:15.

### Definitions and Characteristics

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system (http://www.bioinf.org.uk/abs/#cdrdef).

The term “antigen binding fragment” of an antibody, as used herein, refers to one or more fragments of an intact antibody that retain the ability to specifically binds to a the βig-h3 antigen. Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)₂ fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH:VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8(10); 1057-1062 and U.S. Pat. No. 5,641,870).

In one embodiment, the antibody fragment of the invention is an antigen binding fragment selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL or VH domains as disclosed herein.

The Fab of the present invention can be obtained by treating an antibody which specifically reacts with βig-h3 with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with βig-h3 with a protease, pepsin. Also, the F(ab′)2 can be produced by binding Fab′ described below via a thioether bond or a disulfide bond.

The Fab′ of the present invention can be obtained treating F(ab′)2 which specifically reacts with βig-h3 with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. Nos. 5,859,205; 5,585,089; 4,816,567; EP0173494).

The humanized monoclonal antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred. Examples of tandem type humanized antibody expression vector include pKANTEX93 (WO 97/10354), pEE18 and the like. Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

As used herein, the term K_{D} is intended to refer to the dissociation constant, which is obtained from the ratio of Kd to Ka (i.e. Kd/Ka) and is expressed as a molar concentration (M). K_{D} values for antibodies can be determined using methods well established in the Art. One method for determining the K_{D} of an antibody is by using SPR, especially using a biosensor system such as a Biacore® system in the conditions described in the part Methods of measure.

The term “k_{d}” (sec⁻¹), as used herein, refers to the dissociation rate constant of a particular Ab-antigen interaction ([Ab] [antigen]/[Ab-antigen complex]). Said value is also referred to as the k_{off} value.

The term “kₐ” (M⁻¹×sec⁻¹), as used herein, refers to the association rate constant of a particular Ab-antigen interaction and is the reciprocal of the k_{d}.

The term “K_{D}” (M), as used herein, refers to the dissociation equilibrium constant of a particular Ab-antigen interaction and is obtained by dividing the k_{d} by the kₐ.

The term “K_{A}” (M⁻¹), as used herein, refers to the association equilibrium constant of a particular Ab-antigen interaction and is obtained by dividing the kₐ by the k_{d}.

As used herein, the thermostability is assessed by Differential Scanning Calorimetry (DSC). It is measured as described in the part Methods of measure.

A “humanized antibody” or “chimeric antibody” shall mean an antibody derived from the parent murine antibody by the methods available to the skilled person and for example, those disclosed herein. Preferably, a “humanized antibody” or “chimeric antibody”, or an antigen-binding fragment thereof, will comprise the set of 6 CDRs of the murine antibody m18B3, possibly with mutations in the CDRs.

The humanized antibodies, as the chimeric one, and the antigen-binding fragments, retain or substantially retain the antigen-binding properties of the parental murine antibody m18B3, and, as disclosed herein, the humanization may confer interesting and unexpected functionalities with respect to the murine and/or chimeric 18B3 monoclonal antibodies.

The CDRs or some of them may differ from the murine CDRs following SDR approaches (super-grafting) or other useful methods. The humanization described herein allows providing the monoclonal antibodies and antigen-binding fragments thereof with interesting and unexpected functionalities, as discloses herein, especially affinity, dissociation rate, thermal stability (DSC), combined with functional properties for therapeutic use. H-330 and H-311 were shown to be very attractive, and L-41 and L-218 as well. The skilled person could be able to introduce amino acid changes (e.g. up to 1, 2, 3, 4, 4, 5, 6, 7, 8, 9, 10 amino acids) into these VH and VL regions, without substantially changing some of these functionalities and the functional properties. VH and/or VL domains so changed would still be encompassed by the definition of these VH and VL domains.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGI, IgG2, IgG3 and IgG4. Preferably, IgG1 is used.

“Treatment” or “therapy” refer to both therapeutic treatment and prophylactic or preventative measures. Preferably, it is therapeutic treatment.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human. Except indicated to the contrary, the terms “subject”, “patient” and the like include mammals including human.

The terms “cancer” and “cancerous” refer to or describe the physiological condition in mammals that is typically characterized by unregulated cell growth.

The term “nucleic acid” or “oligonucleotide” or grammatical equivalents herein refer to at least two nucleotides covalent linked together. A nucleic acid of the present invention is preferably single-stranded or double stranded and will generally contain phosphodiester bonds.

Amino acid sequence “variants” (or mutants) of the antibody are prepared by introducing appropriate nucleotide changes into the antibody DNA, or by nucleotide synthesis. Such modifications can be performed, however, only in a very limited range, e.g. as described herein. For example, the modifications do not alter the above-mentioned antibody characteristics such as the IgG isotype and antigen-binding, but may improve the yield of the recombinant production, protein stability or facilitate the purification.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98% or 99%, sequence identity to the native (endogenous) nucleotide sequence.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 95, 100%, or any amount of reduction in between as compared to native or control levels.

### Compositions and Pharmaceutical Compositions

Another object of the invention is a composition or a pharmaceutical composition comprising at least one Hz monoclonal antibody or antigen-binding fragment thereof, as disclosed and provided herein. The composition may further comprise a vehicle or diluent, in particular a vehicle or diluent suited to the intended use of the antibody. If the composition is a pharmaceutical composition, use is made of a pharmaceutically acceptable carrier, diluent or

The pharmaceutical compositions may comprise (i) at least one humanized anti-βig-h3 monoclonal antibody or an antigen-binding fragment thereof, according to the invention, and (ii) at least one additional anti-tumoral medicament, such as an antibody directed against another target and/or a chemotherapeutic drug (such as small molecule). Both active principles may be present in the same composition. Or, these at least two active principles are separated, e.g. in separate vials or compositions. In an aspect, the composition comprises the at least two active principles for use in treating cancers, as described herein, and/or for use in modulating immunity, for a simultaneous, separate or sequential administration to a mammal, including man.

As additional active principle, one may cite in particular doxorubicine, gemcitabine, camptothecin, paclitaxel. It may also be another antibody. The other antibody may be selected from the group consisting of another cancer marker or receptor, another antigen expressed on immune competent cells, an immune checkpoint, and a combination thereof.

Pharmaceutically acceptable carriers or excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be performed in a rectal suppository formulation (see above) or in a suitable enema formulation. Patches may also be used. The compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

For example, an antibody present in a pharmaceutical composition of this invention can be supplied at a concentration of 10 mg/mL in either 100 mg (10 mL) or 500 mg (50 mL) single-use vials. The product may be formulated for IV administration in 9.0 mg/mL sodium chloride, 7.35 mg/mL sodium citrate dihydrate, 0.7 mg/mL polysorbate 80, and Sterile Water for Injection. The pH may be adjusted to 6.5.

A pharmaceutical composition of the invention for injection (e.g., intramuscular, i.v.) could be prepared to contain a pharmaceutically acceptable carrier, diluent or excipient, e.g. sterile buffered water (e.g. 1 ml for intramuscular), and between about 1 ng to about 100 mg, e.g. about 50 ng to about 30 mg or more preferably, about 5 mg to about 25 mg, of the antibody according to the invention.

In certain embodiments, the use of liposomes and/or nanoparticles is contemplated for the introduction of antibodies into host cells. The formation and use of liposomes and/or nanoparticles are known to those of skill in the art. Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

For Use, Methods of Use (e.g. Therapeutic Treatment), Use for the Manufacture

Unless inappropriate, all the features disclosed herein apply to the different objects of the invention, such as “for use”, “methods of use” or “of treating”, “use for the manufacture a medicament”. In an embodiment, the patient or subject is a mammal, preferably a human.

Another object of the invention is a humanized anti-βig-h3 monoclonal antibody or an antigen-binding fragment thereof, or a pharmaceutical composition, as disclosed herein, for use as a medicament.

In an aspect, the present invention relates to such humanized anti-βig-h3 monoclonal antibody or antigen-binding fragment thereof, or compositions containing the same, for use (i) in treating a solid cancer or (2i) as an immunomodulating composition. In particular, the immunomodulating effect may be helpful in treating or in the course of treating a cancer. Immunomodulating may comprise restoring or activating the CD8+ T cell activity.

In another aspect, the present invention relates to such humanized anti-βig-h3 monoclonal antibody or antigen-binding fragment thereof, or compositions containing the same, for use to decrease the rigidity of the stroma. Especially, this function allows access to the tumor to other anti-tumoral medicaments, such as antibodies, monoclonal antibodies.

Thus, generally speaking, the present invention relates to such humanized anti-βig-h3 monoclonal antibody or antigen-binding fragment thereof, or compositions containing the same, for use in treating a solid cancer.

In an embodiment, the solid tumor is one wherein βig-h3 is expressed at the stroma.

In preferred embodiment, the solid tumor may be, or is selected from the list consisting of, breast cancer, uterine/cervical cancer, oesophageal cancer, pancreatic cancer, colon cancer, colorectal cancer, kidney cancer, ovarian cancer, prostate cancer, head and neck cancer, non small cell lung cancer stomach cancer, tumors of mesenchymal origin (i.e; fibrosarcoma and rhabdomyosarcoma) tumors of the central and peripheral nervous system (i.e; including astrocytoma, neuroblastoma, glioma, glioblastoma) thyroid cancer. Preferably the solid tumor pancreatic cancer eosophage squamous cell carcinoma, gastric and hepatic carcinoma, colon cancer, or melanoma. In a preferred embodiment the solid tumor is a pancreatic cancer. More preferably the pancreatic cancer is pancreatic ductal adenocarcinoma.

The present invention also relates to a method of treatment of a solid cancer, comprising administering to a patient in need thereof a sufficient amount of such an antibody or antigen-binding fragment thereof, or a pharmaceutical composition comprising the same.

The present invention also relates to a method of immunomodulation, comprising administering to a patient in need thereof a sufficient amount of humanized anti-βig-h3 monoclonal antibody or antigen-binding fragment thereof, or of such a pharmaceutical or immunomodulatory composition. The antibody or fragment may help restoring or activating the CD8+ T cell activity.

As used herein, the terms “treatment” and “treat” refer to curative or disease modifying treatment, including treatment of subjects who have cancer, or is diagnosed as suffering from cancer, especially a cancer wherein the stroma expresses βig-h3, and includes suppression of clinical relapse. The treatment may concern a subject having a cancer, in order to cure, delay the onset of, reduce the severity of, or ameliorate one or more symptoms of said cancer, or in order to prolong the survival of a subject beyond that expected in the absence of such treatment.

The disclosed antibodies or antigen-binding fragments thereof can be administered as a therapeutic agent to a subject, in particular a human, in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 0.5 mg/kg to about 25 mg/kg, or from about 0.5 to about 10, 5, 3 or 2 mg/kg of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labelled antibody of the present invention, or an antigen-binding fragment thereof. If desired, an effective daily dose of a pharmaceutical composition may be administered as two, three, four, five, six or more sub-doses administered separately at appropriate intervals throughout the day, optionally, in unit dosage forms. In some embodiments, the monoclonal antibodies of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided to a subject, especially a human, as a daily dosage of an antibody of the present invention or an antigen-binding fragment thereof in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

### Combination

The present invention also provides for therapeutic applications or methods of treatment where an antibody of the present invention or an antigen-binding fragment thereof is used in combination with at least one further therapeutic agent, e.g. for treating cancer. Such administration may be simultaneous, separate or sequential; i.e. treatment with the two active principles can be at the same time (e.g. simultaneously or concurrently), or at different times (e.g. consecutively or sequentially), or a combination thereof. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogens, cell cycle control/apoptosis regulating agents, hormonal regulating agents, and other agents described below.

In some embodiments, the antibody of the present invention or an antigen-binding fragment thereof is used in combination with a chemotherapeutic agent or an antibody, especially monoclonal antibody specifically targeting a tumor antigen, receptor or ligand, such as an ICI. The term “chemotherapeutic agent” refers to chemical compounds that are effective in inhibiting tumor growth.

Therefore, the present invention provides a combination of
i. a humanized antibody or antigen-fragment thereof as disclosed herein; and
ii. a therapeutic agent for treating cancer, as described herein;
for simultaneous or sequential use in the treatment of a solid tumor.

In particular, the present invention provides a combination of
i. a humanized antibody or antigen-fragment thereof as disclosed herein; and
ii. an immune checkpoint inhibitor (ICI);
for simultaneous or sequential use in the treatment of a solid tumor.

Typically, the checkpoint blockade cancer immunotherapy agent is an antibody. In some embodiments, the checkpoint blockade cancer immunotherapy agent is an antibody selected from the group consisting of anti-CTLA4 antibodies, anti-PDI antibodies, anti-PDLI antibodies, anti-PDL2 antibodies, anti-TIM-3 antibodies, anti-LAG3 antibodies, anti-IDOI antibodies, anti-TIGIT antibodies, anti-B7H3 antibodies, anti-B7H4 antibodies, anti-BTLA antibodies, and anti-B7H6 antibodies. These antibodies are preferably monoclonal antibodies or antigen-binding fragments thereof. The antibody may be in particular the PD-I blocking antibody Pembrolizumab, Nivolumab, Atezolizumab, Avelumab or Durvalumab, or the CTLA-4 blocking antibody Ipilimumab. This combination is based on disclosure of WO2020079164, which describes the combination of ch18B3 and an ICI. This document is incorporated herein by reference.

Targeting βig-h3 protein with the antibodies of the present invention or an antigen-binding fragment thereof in combination with existing chemotherapeutic treatments will be more effective in killing the tumor cells than chemotherapy alone. Examples include, but are not limited to, cisplatin, taxol, etoposide, mitoxantrone, actinomycin D, campthotecin, methotrexate, gemcitabine, mitomycin, dacarbazine, 5-fluorouracil, doxorubicine and daunomycin.

The antibodies of the invention or antigen-binding fragments thereof may be used in combination with Immune Check Point Inhibitors as further anti-cancer agent, such as anti-PD1, anti-PD-L1 or anti-CTLA4 antibodies.

In one method of the invention, the βig-h3-binding antibody or fragment is administered to the patient prior to administration of a second anti-cancer agent.

### Production of Antibodies

The antibodies of the present invention and antigen-binding fragments thereof are produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

Mammalian cells are the preferred hosts for production of therapeutic antibodies, due to their capability to glycosylate proteins in the most compatible form for human applications. Bacteria very rarely glycosylate proteins, and like other type of common hosts, such as yeasts, filamentous fungi, insect and plant cells yield glycosylation patterns associated with rapid clearance from the blood stream. Among mammalian cells, Chinese hamster ovary (CHO) cells are the most commonly used. In addition to giving suitable glycosylation patterns, these cells allow consistent generation of genetically stable, highly productive clonal cell lines. They can be cultured to high densities in simple bioreactors using serum-free media, and permit the development of safe and reproducible bioprocesses. Other commonly used animal cells include baby hamster kidney (BHK) cells, NSO- and SP2/0-mouse myeloma cells.

In an embodiment, the antibodies according to the invention are produced or expressed in mammal cells, preferably wild-type mammal cells, preferably of rodent origin, especially CHO cells.

Modifications and changes may be made in the structure of an antibody of the present invention and still obtain a molecule having like characteristics. For example, certain amino acids can be substituted for other amino acids in a sequence without appreciable loss of activity. Because it is the interactive capacity and nature of an antibody that defines that antibody's biological functional activity, certain amino acid sequence substitutions can be made in an antibody sequence (or, of course, its underlying DNA coding sequence) and nevertheless obtain an antibody with like properties. In making such changes, the hydropathic index of amino acids can be considered. The importance of the hydropathic amino acid index in conferring interactive biologic function on an antibody is generally understood in the art. It is known that certain amino acids can be substituted for other amino acids having a similar hydropathic index or score and still result in an antibody with similar biological activity. Each amino acid has been assigned a hydropathic index on the basis of its hydrophobicity and charge characteristics.

It is believed that the relative hydropathic character of the amino acid determines the secondary structure of the resultant antibody, which in turn defines the interaction of the antibody with other molecules, for example, enzymes, substrates, receptors, antibodies, antigens, and the like. It is known in the art that an amino acid may be substituted by another amino acid having a similar hydropathic index and still obtain a biologically functionally equivalent polypeptide. In such changes, the substitution of amino acids whose hydropathic indices are within ±2 is preferred, those which are within ±1 are particularly preferred, and those within +0.5 are even more particularly preferred.

Substitution of like amino acids can also be made on the basis of hydrophilicity, particularly where the biologically functionally equivalent peptide or polypeptide thereby created is intended for use in immunological embodiments. U.S. Pat. No. 4,554,101, incorporated herein by reference or to which the person skilled in the art: may refer, states that the greatest local average hydrophilicity of a polypeptide, as governed by the hydrophilicity of its adjacent amino acids, correlate with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent, polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Amino acid substitution may be chosen or selected differently. Possible substitutions have been documented in WO99/51642, WO2007024249 and WO2007106707.

### Nucleic Acids and Vectors

The isolated nucleic acid sequences disclosed and provided herein are also object of the invention. Thus the invention also relates to an isolated nucleotide sequence selected from the group consisting of the nucleotide sequences SEQ ID NO: 21, 22, 23 and 24 and more particularly combinations or sets of two nucleotide sequences separated or linked together: SEQ ID NO: 21 and 23, 21 and 24, 22 and 23, 22 and 24. A nucleotide sequence encoding H-330 V1.2 is as set forth in SEQ ID NO: 29 and may be used in place of SEQ ID NO: 21 to produce the monoclonal antibodies when the mutated version C102S is wished.

As mentioned above, methods for producing the antibodies are known from the person skilled in the art. The mammal cells, preferably rodent cells such as CHO cells, preferably wild-type cells are transfected with one or several expression vectors. Preferably, the cells are co-transfected with an expression vector for light chain and with an expression vector for heavy chain. Cell transfection is also known from the person skilled in the art. As transfection that may be performed, one may mention without limitation standard transfection procedures, well-known from the man skilled in the art, such as calcium phosphate precipitation, DEAE-Dextran mediated transfection, electroporation, magnetofection, nucleofection (AMAXA Gmbh, GE), liposome-mediated transfection (using Dreamfect®, Lipofectin® or Lipofectamine® technology for example) or microinjection.

Expression vectors are known. As vectors that may be used, one may mention without limitation: pcDNA3.3, pOptiVEC, pFUSE, pMCMVHE, pMONO, pSPORT1, pcDV1, pcDNA3, pcDNA1, pRc/CMV, pSEC. One may use a single expression vector or several expression vectors expressing different parts of the antibody.

The invention also relates to an expression vector encoding a Heavy Chain of an Hz antibody of the invention, an expression vector encoding a Light Chain of an Hz antibody of the invention, or an expression vector encoding a Heavy Chain and a Light Chain of such an Hz antibody.

Another object of the invention is a host cell containing a vector or a set of vectors of the invention. The host cell may be a mammal cell, preferably a rodent cell, more preferably CHO cell. Still more preferably, the host cell may be a wild-type mammal cell, preferably a wild-type rodent cell, most preferably a wild-type CHO cell.

The person skilled in the art fully owns the methods to generate the antibodies according to the invention using such a vector or vectors and cells such as CHO cells.

### Methods of Measure as Used Herein (See Also Examples for Completeness)

Affinity and dissociation rate

| | |
|---|---|
| Instrument: | Biacore T200 (Cytiva) |
| Temperature: | 25° C. analysis temperature, 12° C. sample compartment temperature |
| Sensor chips: | Series S Sensor Chip C1 (Cytiva, order number 29104944) |
| | Series S Sensor Chip CM5 (Cytiva, order number 29104988) |
| Flow cells (fc): | fc1: reference |
| | fc2: ligand immobilized (captured) |
| | fc3: reference |
| | fc4: ligand immobilized (captured) |
| Detection: | fc2-1 and fc4-3; or fc2-1, fc3-1, fc4-1 |
| Analysis buffer: | A) 10 mM HEPES (pH 7.4), 150 mM NaCl, 3 mM EDTA, 0.05% |
| | Tween 20, 0.22 μm filtered |
| | B) 10 mM HEPES (pH 7.4), 300 mM NaCl, 3 mM EDTA, 0.05% |
| | Tween 20, 0.25 mg/mL BSA, 0.22 μm filtered |
| Flow rates: | 10 μL/min during capturing of ligand (reversible |
| | immobilization) |
| | 50 μL/min during interaction analysis of analyte (association |
| | and dissociation) |
| | 20 μL/min during surface regeneration |
| Association: | 3 min |
| Dissociation: | up to 15 min |
| Regeneration: | Anti-mouse IgG: Injection of 10 mM glycine pH 1.7 (3 min, |
| | 20 μL/min) |
| | Anti-human IgG: Injection of 3M MgCl₂ (1 min, 20 μL/min) |

### DSC

Differential Scanning Calorimetry (DSC) is an analysis technique used to characterize the stability of a protein or other biomolecule directly in its native form. It does this by measuring the heat change associated with the molecule's thermal denaturation when heated at a constant rate.

DSC protocol used herein:
Microcal™ VP-Capillary DSC system was used to perform differential scanning calorimetry experiments.
Samples were stored at −20° C. After thawing all samples were centrifuged (20.000 g, 5 min, 4° C.), and when necessary diluted in PBS to a concentration of 1 mg/mL. Their protein content was quantitated Nanodrop ND-1000 spectrophotometer (s/n: 4847) with IgG analysis program, prior to DSC analysis.
The pre-equilibration time was 3 min and the thermograms that followed were acquired between 2° and 110° C. with a scanning rate of 60° C./hour, a filtering period of 25 sec and medium feedback. Prior to sample analysis, 5 buffer/buffer scans were measured to stabilize the instrument, and a buffer/buffer scan was performed between each protein/buffer scan.
The data was fitted to a non-2-state unfolding model, with the pre- and post-transition adjusted baseline subtracted. The calorimetric enthalpy (ΔH) is determined as the area under the peak of the transition, whereas the van't Hoff enthalpy (ΔHv) is determined from the model used. Red solid line represents the measured data, black solid line represents the best fit for the model used and the gray lines represent the contributions from a single unfolding unit to the overall protein unfolding.
DSC general considerations:
   Tm or denaturation/melting temperature is the point at which the concentration of the unfolded and folded species is equal and is the midpoint of the unfolding transition. As a parameter it describes the susceptibility of the protein to thermal denaturation and thus it relates to the stability of the protein ⋄ The higher the Tm the more stable the protein.
   Tonset is the temperature at which the unfolding transition begins. The values for this parameter are usually 5 to 10° C. lower than the Tm. It is also a parameter describing protein stability, but with relevance to the resistance of thermal denaturation.
   T1/2 is the width of the transition at half height of the peak. It describes the broadness of the transition with typical values between 1 to 15° C. The parameter correlates with compactness of protein packing, where a broader transition corresponds to a less compact protein.
   ΔH is the calorimetric enthalpy of unfolding and reflects the disruption of intramolecular interactions of the protein (i.e. breaking intra- and inter-domain interactions). The process is endothermic thus giving positive enthalpic values.
   Total Area is the entire enthalpy of unfolding (total area under the thermogram) and it reflects thermodynamic stability.
   Monoclonal antibodies typically show a complex, multi-domain thermogram. The largest, most prominent domain peak is the Fab of the antibody. The CH2 and/or CH3 domains are also commonly seen. The relative positions and TMs of the different domains depend on the specific monoclonal antibody, and can vary depending on subclass and engineering.

### Transient Transfection in CHO Cells

The humanized “V1 version” antibodies were produced in CHO cells. CHO DG44 cells were maintained in ventilated Erlenmeyers (Corning) at 37° C. and 5% CO2 on an orbital shaker. The day before transfection, cells were passaged at a defined density (according to MO.CEL.120). On transfection day (day 0), cells were mixed to the transfection reagent and plasmid DNA for the production of a pilot lot (30 ml).

The present invention will now be described using non limiting examples referring to the figures

### BRIEF DESCRIPTION OF THE DRAWINGS

FIG. 1. Schematic representation of the structure of human βig-h3. 18B3 mAb recognizes the 549-558 epitope which comprises YH18 domain important for αvβ3 and collagen binding.

FIG. 2. Graph showing ELISA results for the chimeric 18B3 (considered as the reference mAb) as well as the 4 humanized variants. Mean of 6 experiments (+Standard Deviation).

FIG. 3. Graph showing cytotoxic CD8+ T cells activation and proliferation for the chimeric 18B3 (considered as the reference mAb) as well as the 4 humanized variants. Mean of 3 experiments (+Standard Deviation). The statistical significance of the parameters is assessed through Student t-test and One-Way Anova done by using GraphPad Prism Software.

FIG. 4. Graph showing tumor weight of sc implanted tumor pancreatic cancer tumor cells in presence of ctrl IgG1 Ab, chimeric 18B3 (considered as the reference mAb) as well as the 4 humanized variants. Tumor cells are embedded as plugs in a Matrigel 1:1 mix (Corning) and subcutaneously injected into the flanks of normal C57BL6 mice together with 6 μg of humanized versions mAb per mouse. The control consists in an irrelevant isotype control IgG1 mAb. Same mice population (n=5) is used for each quantity to be evaluated. The tumor grafts are dissociated and the quantity of tumoral cells in the graft are then assessed by FACS staining at 4° C. and analyzed with the FlowJo software. The statistical significance of the parameters is assessed through Student t-test and One-Way Anova done by using GraphPad Prism Software.

FIG. 5. Graph showing number of tumor cells in of sc grafts in presence of ctrl IgG1 Ab, chimeric 18B3 (considered as the reference mAb) as well as the 4 humanized variants. Tumor cells are embedded as plugs in a Matrigel 1:1 mix (Corning) and subcutaneously injected into the flanks of normal C57BL6 mice together with 6 mg of humanized versions mAb per mouse. The control consists in an irrelevant isotype control IgG1 mAb. Same mice population (n=5) is used for each quantity to be evaluated. The tumor grafts are dissociated and the quantity of tumoral cells in the graft are then assessed by FACS staining at 4° C. and analyzed with the FlowJo software. The statistical significance of the parameters is assessed through Student t-test and One-Way Anova done by using GraphPad Prism Software.

FIG. 6. Graph showing number of non activated CD8 T cells in of sc grafts in presence of ctrl IgG1 Ab, chimeric 18B3 (considered as the reference mAb) as well as the 4 humanized variants. Tumor cells are embedded as plugs in a Matrigel 1:1 mix (Corning) and subcutaneously injected into the flanks of normal C57BL6 mice together with 6 mg of humanized versions mAb per mouse. The control consists in an irrelevant isotype control IgG1 mAb. Same mice population (n=5) is used for each quantity to be evaluated. The tumor grafts are dissociated and the quantity of tumoral cells in the graft are then assessed by FACS staining at 4° C. and analyzed with the FlowJo software. The statistical significance of the parameters is assessed through Student t-test and One-Way Anova done by using GraphPad Prism Software.

FIG. 7. Graph showing number of tumor cells in of sc grafts in presence of ctrl IgG1 Ab, as well as the 2 humanized variants (H330/L41 and H330/L228) in the original version (V1) and in the mutated version (V1.2) on C102. On both mAbs, the implementation of a mutation in position 102 of the heavy chain 330 (replacement of cysteine residue by serine) was done, in order to minimize the risk of Post Translational Modifications (PTMs).

Tumor cells are embedded as plugs in a Matrigel 1:1 mix (Corning) and subcutaneously injected into the flanks of normal C57BL6 mice together with 6 mg of humanized versions mAb per mouse. The control consists in an irrelevant isotype control IgG1 mAb. Same mice population (n=5) is used for each quantity to be evaluated. The tumor grafts are dissociated and the quantity of tumoral cells in the graft are then assessed by FACS staining at 4° C. and analyzed with the FlowJo software. The statistical significance of the parameters is assessed through Student t-test and One-Way Anova done by using GraphPad Prism Software.

FIG. 8. Graph showing number of activated CD8 T cells in of sc grafts in presence of ctrl IgG1 Ab as well as the 2 humanized variants (H330/L41 and H330/L228) in the original version (V1) and in the mutated version (V1.2) on C102. Tumor cells are embedded as plugs in a Matrigel 1:1 mix (Corning) and subcutaneously injected into the flanks of normal C57BL6 mice together with 6 mg of humanized versions mAb per mouse. The control consists in an irrelevant isotype control IgG1 mAb. Same mice population (n=5) is used for each quantity to be evaluated. The tumor grafts are dissociated and the quantity of tumoral cells in the graft are then assessed by FACS staining at 4° C. and analyzed with the FlowJo software. The statistical significance of the parameters is assessed through Student t-test and One-Way Anova done by using GraphPad Prism Software.

### EXAMPLES

### Example 1: Epitope Mapping

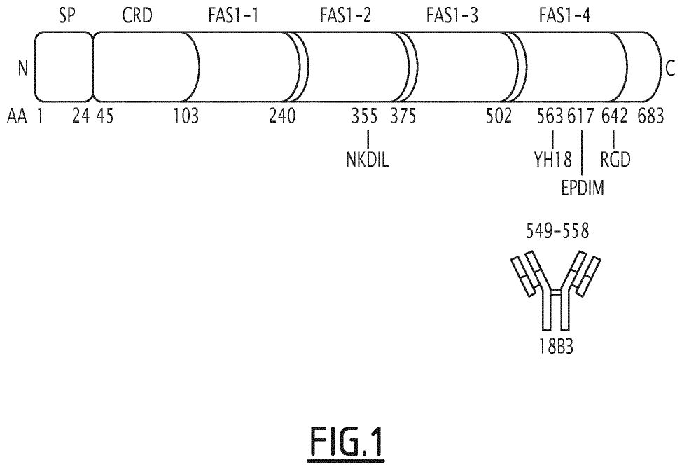

Epitope mapping studies showed that this antibody targets the FAS1 4^{th} domain of the βig-h3 protein (linear epitope ALPPRERSRL) of the βig-h3-protein (AA residues 549-558). See FIG. 1. It has been possible to restrict further the epitope to LPPRERSR.

### Example 2

The Proof of Concept (PoC) phase is aiming at demonstrating that the 18B3 mAb (Bae et al., 2014 Acta Physiol 2014, 212, 306-315) directed against the βig-h3 protein can play a key role in cancer treatment by efficiently and specifically depleting the βig-h3 protein, thus (i) increasing the cytotoxic activity of CD8⁺ T cells, and (ii) reducing the rigidity of the stroma, which restores the immune system's accessibility to the tumor, ultimately leading to significant tumor reduction and survival rate in mice.

To that aim, significant number of experiments were conducted both in-vitro and in-vivo (in relevant mice models). It is to be noticed that most of the experiments were conducted in-vivo, showing evidence that an anti-βig-h3 therapeutic mAb could be effective, standalone or in combination, in addressing PDAC and potentially other Cancers.

In a nutshell, the conclusions of this PoC phase are as following:
1. In PDAC model, the targeted depletion of the βig-h3 protein, by the 18B3 mAb, allows for:
   a. limiting the tumor growth,
   b. restoring the cytotoxic activity of CD8+ T lymphocytes,
   c. reducing the rigidity of the stroma,

This has been demonstrated in both in-vitro and in-vivo assays specifically developed by the scientific team to that purpose.
2. Experiments performed using 2 different mice strains developing pancreatic tumors showed that population treated for 3 weeks with twice-a-week subcutaneous injection of the 18B3 have significant tumor reduction and increased survival rate.
3. Several experiments done at higher dose of 18B3 showed that increasing the concentration of the mAb minimize further the tumor growth and may increase mice survival.
4. On mice HSG ovarian and bladder cancer, respectively, we have demonstrated that 18B3 mAb treatment significantly reduces tumor growth. In the anti-PD1 resistant bladder cancer, the 18B3 mAb is shown to be a potential complement or alternative to the anti-PD1.

The overall conclusion of this comprehensive set of data is that the concept of using a βig-h3 protein depleting mAb for therapeutic use is confirmed.

### Example 3: Chimerization and Humanization of the 18B3 Murine mAb

### 1. Method

The project was conducted in 4 phases, as following:
Phase 1: sequencing of the murine 18B3 mAb
Phase 2: chimerization of the murine 18B3 mAb
Phase 3: design of Humanized variants by CDR Grafting (antibody reshaping)
Phase 4: production and analytical testing of the humanized variants

### Phase 1: Sequencing of the Murine 18B3 mAb

The sequence of the mouse 18B3 (Isotype IgG1/k) VH and VL domains was performed from hybridoma cells using cDNA sequencing methodology.

To that aim, the RNA was extracted from the hydridoma cells. Corresponding DNA strand was synthetized by high fidelity RT-PCR, following by the second strand to get the double-stranded cDNA. cDNA was then sequenced and translated into the proteic sequence.

VH sequence: SEQ ID NO: 17

VL sequence: SEQ ID NO: 18

### Phase 2: Chimerization of the 18B3 mAb

The Chimerization consists in replacing the constant domains of the mouse 18B3 mAb by a human sequence. Sequences encoding the variable domain of heavy chain (VH SEQ ID NO: 19) and the variable domain of light chain (VL SEQ ID NO: 20) were optimized for expression in mammalian cells and synthetized. The corresponding synthetic genes were then cloned into a vector system that contains the human constant regions of IgG1 heavy chain (SEQ ID NO: 25) and kappa light chain (SEQ ID NO: 26). Once validated by sequencing, the vectors were amplified for the preparation of low-endotoxin plasmid DNA, which was again verified by sequencing.

The chimeric mAb was then produced in CHO mammalian cells by transient expression of the plasmid, then purified: CHO DG44 cells were maintained in ventilated Erlenmeyers (Corning) at 37° C. and 5% CO₂ on an orbital shaker. The day before transfection, cells were passaged at a defined density. On transfection day (day 0), DNA plasmids encoding the light and the heavy chains of 18B3 chimeric antibody were added to the cell suspension mixed to the transfection reagent for the production of a pilot lot.

The supernatant was purified by protein A affinity chromatography. After dialysis in PBS and sterile filtration (0.22 μm), the total protein concentration was determined by spectrophotometric reading at 280 nm. The purified Chimeric mAb was then store at temperature ≤−20° C.

The chimeric mAb was tested for integrity by SDS-PAGE. The affinity to ligand of the Chimeric was compared to the parental mouse 18B3 mAb by ELISA.
For the ELISA:
The antigen (recombinant human βIG-H3: rhβIG-H3, R&D systems, cat. 3409-BG, lot NDM061911A) was coated at 1 μg/ml overnight at 4° C.,
The antigen was removed and non-specific sites were blocked using PBS-Milk at 2.5% for 1 h at RT
The mAb to be tested (anti-hβIG-h3 18B3 parental (murine) or chimeric) was added from 10 μg/ml, then diluted 10× on 7 wells and incubated 2 hours at room temperature,
After removal and washing of the plate, the secondary antibody was added: anti-murine IgG conjugated to HRP for the murine 18B3 (parental) and anti-human IgG (Fc specific) conjugated to HRP for the chimeric 18B3 and incubated 2 hours at room temperature,

Uncolored substrate (TMB: 3,3′,5,5′-tetramethylbenzidine) was added and turn out to blue under the action of the HRP. The colorimetric signal is proportional to the amount of mAb bound onto the antigen.

The reaction was stopped by adding sulfuric acid and TMB turns then to yellow. The quantity of mAb was assessed by spectrophotometry (optical density) at 450 nm.

The chimerization was successful with very similar biophysical characteristics as compared to the parental murine antibody.

### Phase 3: Design of the Humanized 18B3 Variants by CDR Grafting

The objective of this phase is to get several variants of the chimeric 18B3 that have been humanized further in order to reduce immunogenicity of the mAb in Humans and increase its half-life. It is considered that the % of Humanness should be above 85%.

The humanization was performed using the CDR-grafting technology.

The humanization strategy is based on a combination of technologies, namely:
primary sequence analysis and alignment,
3D-modelling,
selection of best human germlines.

Indeed, the combination of a structural (3D) model with pure sequence analysis allowed discriminating potentially between real paratope-facing and non-paratropic residues in the CDR regions. In addition, the structural models permit to drive the choices regarding back-mutations in light of the selected germline backbones used, allowing a faster humanization process.

It is to be noticed that the Kabat numbering system was used for the residues identification.
The first step is to select human VH and VL germlines that are as close as possible in sequence to the murine 18B3 mAb.
   For the design of CDR-grafted versions of the anti-HuβIG-H3 18B3 murine VH, three human germlines, IGHV3-11*01, IGHV3-30*01 and IGHV1-69*08 were selected (see Table 2). The human germline IGHV3-11*01 was selected because it has a high sequence identity across the whole V-gene with the mAb 18B3-VH; 76.5% (75 identical residues out of a total of 98). Despite the fact that IGHV3-30*01 and IGHV1-69*08 have a lower sequence identity (72.4 and 55% respectively), they were selected because they are widely used germline sequences for human antibody production (according to IMGT/GeneFrequency database) and have other interesting features and provide the possibility of grafting the CDRs in a different molecular environment, especially for IGHV1-69*08. For the N-terminal part of the H-CDR3 and the Framework 4, the human germline IGHJ6*01 (J-GENE) is selected as the closest candidate.
   For the design of CDR-grafted versions of the anti-HuβIG-H3 18B3 murine VL_{K}, three human germlines, IGKV4-1*01, IGKV2-28*01 and IGKV3-15*01, appeared to be the best choices as human framework acceptor regions with percentages sequence identity for all 3 human germlines of 82.2% for IGKV4-1*01, 67.3% for IGKV2-28*01 and 64.4% for IGKV3-15*01. For the N-terminal part of the L-CDR3 and the Framework 4, the human germline IGKJ2*01 (J-GENE) is selected as the closest candidate.
The second step is to graft the CDRs of the 18B3 murine mAb into the selected VH and VL human germlines, without introducing any mutation in the sequence: this is called the V0 version of the humanized variants.
The third step is to optimize the sequence obtained in order (i) to keep the functionality of the humanized variants and (ii) possibly increase the % of humanness. This leads to versions V1 to V3 of the humanized variants. The optimization was conducted as following:
   some amino acid residues in the framework regions (Frs) of the selected human germline variable regions were reverted (back-mutation) to their corresponding murine amino acid residues. Based upon information harvested on the structure of immunoglobulin variable regions, and with the guidance of a molecular model of the mAb 18B3 (VH and VL), these few residues in the Frs were identified as having potential key roles in maintaining the CDRs conformation, or as playing a role in the interface between the variable regions of the heavy chain and the light chain.
   Also, VL and VH are two domains that interact without forming a covalent bond. The interaction between the two domains is maintained through hydrogen and electrostatic bonding. The residues involved in this interaction must also be maintained, otherwise the paratope might be modified and the antibody affinity altered. They were thus kept in the humanized version V1 and V1.2 mutated version C102S.

TABLE 2
below summarizes the results of the % of humanness obtained:

| | Human | | | Humanized versions | | | |
|---|---|---|---|---|---|---|---|
| Chain | Germline | 18B3 | V0 | V | V1.2 | V2 | V3 |
| VH | IGHV3- | 76.5 | 88.8 | 90.8 | — | 94.9 | 98.0 |
| | 11*01 | | | | | | |
| | IGHV3- | 72.4 | 86.8 | 88.8 | 93 | 91.8 | 94.9 |
| | 30*01 | | | | | | |
| | IGHV1- | 55.0 | 75.6 | 80.6 | — | 85.7 | 94.9 |
| | 69*08 | | | | | | |
| VL | IGKV4-1*01 | 82.2 | 96.8 | 96.8 | 96.8 | 100 | 100 |
| | IGKV2- | 67.3 | 90.5 | 91.5 | 91.5 | 95.7 | 97.8 |
| | 28*01 | | | | | | |
| | IGKV3- | 64.4 | 83.5 | 86.1 | 86.1 | 89.3 | 93.6 |
| | 15*01 | | | | | | |

### Phase 4: Manufacturing and Testing of the Humanized Variants

The objective of the phase is to manufacture the selected humanized variants, combining the 3 selected VH and the 3 selected VL, leading thus to 9 different variants.

The production and purification were conducted the exact same way as described for the chimeric, using the VH and VL nucleotide sequences SEQ ID NO: 21-24, as appropriate.

The analytical testing of the 9 manufactured humanized variants was done using several methods:
The % of Humanization achieved by sequence alignment and selection of germlines
The productivity was evaluated by the level of transient expression in mammalian cells.
The antibody affinity was assessed by ELISA as described above (Phase 2 chimerization).
The thermostability assessed by Differential Scanning Calorimetry (DSC): measured using
   Microcal™ VP-Capillary DSC system as described in the Methods of measure above. The purity (aggregation) was checked by High Size Exclusion Chromatography (HP-SEC):
A Shimadzu Prominence HPLC with the following components: CBM-20A system controller, SPD-M20A Photodiode array detector; Column Oven CTO-20A; Autosampler SIL-AC; Pump LC-20AD and degassing unit DGU-20A5.
Column Superdex 200 increase 5/150 GL column from GE Healthcare was used. The column was previously equilibrated in PBS 1×, at 0.25 mL/min, with the column oven set to 30° C.
All samples were centrifuged (20.000×g, 5 min, 4° C.), and had their protein content quantitated by Nanodrop ND-1000 spectrophotometer with IgG analysis program, prior to SEC analysis. When necessary the samples were diluted to a concentration of 1 mg/mL in PBS just before sample injection.
The isocratic program was set to inject about 15 μg of each sample, at 0.25 mL/min during 18 min. After SEC analysis, 280 nm chromatogram was extracted from the raw data, and analyzed by peak integration.
A series of proteins from the GE Lifesciences Molecular Weight SEC Calibration kits were used to calibrate the column in the same conditions and buffer used during the sample analysis. The proteins used were: Aprotinin (6.500 Da), Ribonuclease A (13.700 Da), Carbonic Anhydrase (29.000 Da), Ovalbumin (44.000 Da), Conalbumin (75.000 Da) and Aldolase (158.000). Blue Dextran was used to determine the Void Volume of the column. Column calibration was done according to the Gel Filtration Calibration Kits instructions (GE Lifesciences).
   The production and analytical testing of the 9 variants produces showed that:
All variants have a good productivity, especially variants with Heavy Chain #330 and #311 (to a lesser extent); variants with Heavy Chain #169 are below productivity of the chimeric antibody
The affinity of variants in ELISA assays showed similar binding affinity as compared to the chimeric (and parental in ELISA); nevertheless, the variants using the Heavy Chain #169 are again looking less favorable.
In HP-SEC, all variants showed a very high purity with less than 2 to 3% potential aggregates
In DSC, all variants Tm are above 70° C., the more stable variant being the one using Heavy Chain #330 (Tm above the chimeric antibody and a an unexpected level).

TABLE 3

| Antibody Variant (all | | | | Purity in |
|---|---|---|---|---|
| V1, except V1.2 | Cell Culture | ELISA | DSC | HP-SEC |
| where indicated) | Titer (μg/ml) | Titer (μg/ml) | Fab Tm | (%) |
| H-311 / L-41 | 141 | 0.041 | 77 | 96.83 |
| H-311 / L-228 | 140 | 0.046 | 75.5 | 96.8 |
| H-330 / L-41 | 275 | 0.047 | 82.1 | 97.71 |
| H-330/L-41 V1.2 | 95 | 0.0038 | 83.14 | 98.31 |
| H-330 / L-228 | 249 | 0.040 | 81.7 | 98.09 |
| H-330/L-228 V1.2 | 168 | 0.0036 | 81.98 | 98.57 |
| Chimeric | 178 | 0.030 | 78.9 | 97.05 |
| H-311/L-315 | — | — | 74.7 | — |
| H-330/L-315 | — | — | 82.2 | — |
| H-169/L-41 | — | — | 71.5 | — |
| H-169/L-228 | — | — | 70.5 | — |
| H-169/L-315 | — | — | 73.5 | — |

Reactivities of the 4 produced humanized variants were relatively similar against human βIg-h3. No strong loss of reactivity for the target or degraded behavior regarding biophysical properties were observed:
Variants using the closest germline gene H311-V1 showed reactivity and stability properties similar to the chimeric mAb.
Variants using H330-V1 (especially variants H330-V1/L41-V1 and H330-V1/L228-V1) showed unexpected high Fab Tm, which can be associated with a low tendency to aggregate, and showed reactivity properties similar to the chimeric mAbs.
By analyzing the whole dataset, it appeared that the Heavy chains comprising H-311 or H-330 have a major role for the binding to the target, contrary to the Light chains that do not seem to play a major role for the binding; this makes H-311 or H-330 good choice for combination with different VL domains.
The V1 candidates H-311/L-41 (for using the closest germlines genes for both VH and VL and its general properties) and H-330/L-228 are preferred.
DSC of the H330-V1.2/L41-V1 and H330-V1.2/L228-V1 are still above 80° C., stability properties are similar to unmutated versions (with H-330 V1).

The % of Humanness obtained in V1 candidates is very good.

Frameworks were slightly modified to increase humanness while keeping AA residues known to be involved in the conformation (“orientation”) of the CDRs

In the CDRs, 2 mutations were introduced in CDR #2 of VH 330 and VH 311 in positions 57 and 60. The 3D modelling showed that these AA are not involved in the paratope and “hidden” in the conformation of the mAb.

TABLE 4
Immunogenicity

| | Chimeric | H311/L41 | H311/L228 | H330/L41 |
|---|---|---|---|---|
| | Value | Value | Value | Value |
| Humanness | >85%* | 90.8/96.8 | 90.8/91.5 | 88.8/96.8 |
| VH/VL (%) | | | | |

| | H330 V1.2/L41 | H330/L228 | H330 V1.2/L228 |
|---|---|---|---|
| | Value | Value | Value |
| Humanness | 89.2/96.8 | 88.8/91.5 | 82/91.5 |
| VH/VL (%) | | | |

All 6 selected variants are exhibiting % of Humanness above the specification, in the range of 89 to 97%, which is a very high score and significantly reduce the risk of immunogenicity in humans moving forward.

### Example 4: ELISA

The analytical method is based on a direct ELISA test.

The antigen (recombinant human βig-h3 protein) is coated onto the surface of 96-well plates. The mAb to be tested is then added

Then, a secondary anti-human mAb conjugated with Horse Radish Peroxidase (HRP) enzyme is added.

Uncolored substrate (TMB: 3,3′,5,5′-tetramethylbenzidine) is added and turn out to blue under the action of the HRP. The colorimetric signal is proportional to the amount of mAb bound onto the antigen.

The reaction is stopped by adding sulfuric acid and TMB turns then to yellow

Quantity of mAb is assessed by spectrophotometry (optical density) at 450 nm.

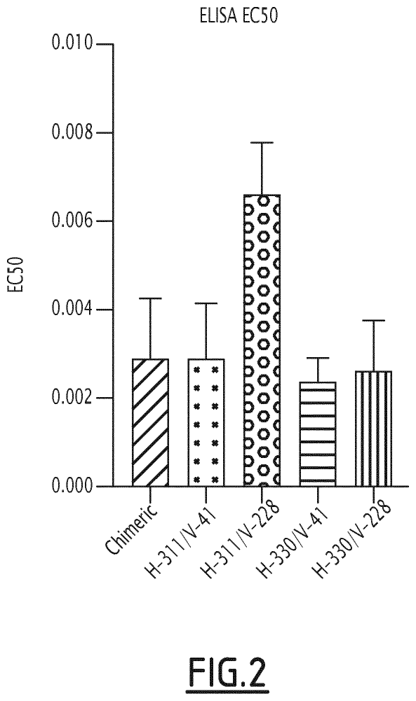

The results obtained are summarized in FIG. 2 for the chimeric 18B3 (considered as the reference mAb) as well as the 4 humanized lead variants. Statistical analysis (one-way ANOVA).

The results obtained showed that the humanized variant H-311/L-228 is showing less affinity to the target as compared to the Chimeric variant. The 3 other humanized variants are not statistically significantly different as compared to the chimeric, showing that the humanization process did not altered the affinity measured by ELISA. Although the 3 variants show a very similar affinity as per EC50 and statistical analysis, the variants with the Heavy chain 330 consistently show the best EC50 (and on average better than Chimeric).

| | |
|---|---|
| Functionality | Functionality defines the ability of the mAb to |
| | be efficient in addressing the expected mechanism |
| | of action, here, in particular: |
| | To decrease the rigidity of the stroma, |
| | To restore the activation of CD8+ T cells pathway |
| | To reduce tumor progression and drastically reduce |
| | the tumoral cells |
| Affinity | Affinity defines the ability of the mAb to bind to |
| | the big-h3 protein. Humanization process keeps the |
| | ability of the mAb in terms of mechanism of action |
| | and efficiency, being similar to or better than to |
| | the parental and chimeric mAb. The affinity constant |
| | (kD) is within the sub-nanomolar range in Biacore |
| Immunogenicity | Immunogenicity defines the ability of the humanized |
| | mAb not to generate an immune response in humans |
| | and thus increase its tolerability and half-life. |
| | This is the objective of the humanization process |
| | as defined by the % of humanness achieved. |
| Stability | Stability relates to the ability of the mAb to remain |
| | structurally (e.g. no aggregation nor degradation) and |
| | functionally efficient in time and within stress |
| | conditions such as temperature, pH, freeze-thaw, . . . |

### Example 5: In Vitro Functional Bioassay

The principle of this test is to evaluate the efficacy of the different mAbs, directed against anti-βig-h3 protein, for their ability to restore Cytotoxic CD8⁺ T cells activation and proliferation, by depleting the protein.

First, freshly spleen-extracted CD8⁺ T cells are put in contact using Antigen Presenting Cells (namely Bone Marrow Derived Cells having processed OVA peptide), to promote activation and proliferation.

Then rhβig-h3 protein (known to block CD8⁺ T cells activation pathway), together with the mAbs to be tested are added.

After 72 hours, the activation and proliferation of the CD8⁺ T cells is quantitatively assessed by Flow cytometry (FACs) analysis.

The efficiency of the mAbs is statistically assessed by comparing level of activation/proliferation as compared to isotype control (irrelevant) mAb.

Bone Derived Marrow Cells are obtained from bone of C57BL6 Wild Type mice and cultivated in vitro for 5 to 6 days. Then, they are maturated by incubation with LPS for 12 hours and activated as antigen presenting cells by adding OVA peptide (SIINFEKEL), which will be processed and presented at the surface of the BMDC (OVA-processed BMDC).

In parallel, CD8⁺ T cells are extracted from spleen and lymph nodes of OT1 mice by crushing and preparation of a single-cell suspension.

OVA-charged BMDC and CD8⁺ T cells are put together with the rh-βig-h3 protein and mAbs to be tested. It is then incubated for 72 hours à 37° C. to allow for activation and proliferation of CD8⁺ T cells.

The proliferation and activation of CD8⁺ T cells was then assessed by FACS staining at 4° C. and analyzed with the FlowJo software.

The statistical significance of the proliferation is assessed through Student t-test and One-Way Anova done by using GraphPad Prism Software.

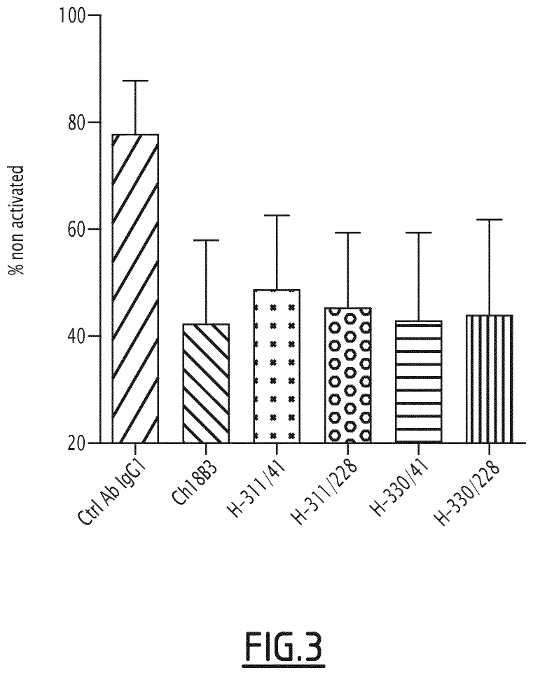

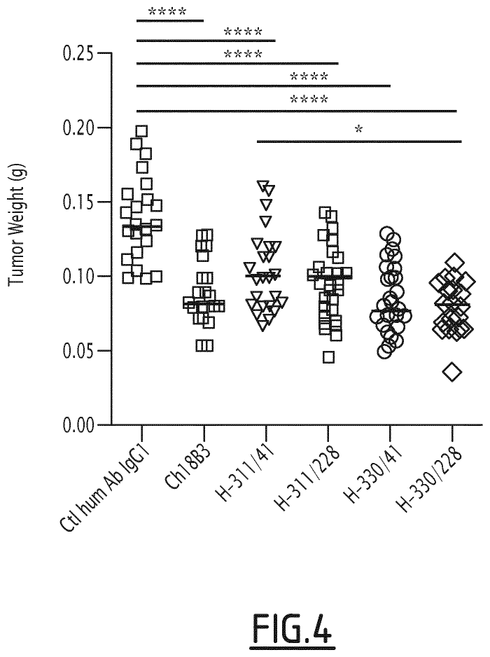

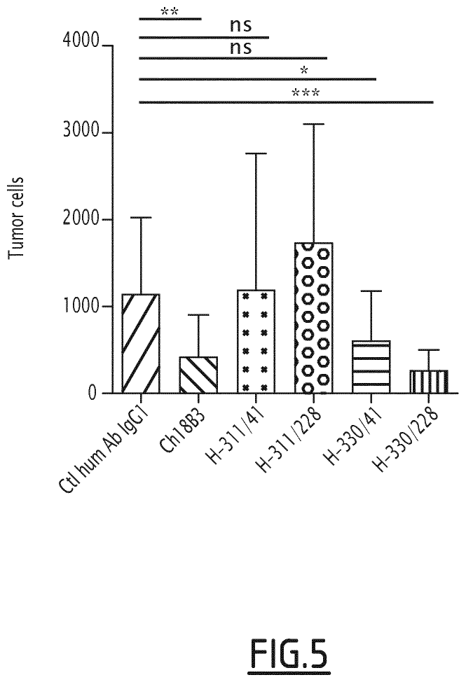

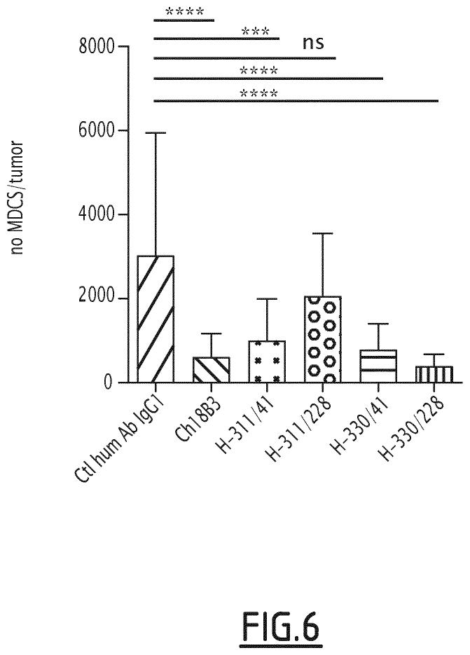

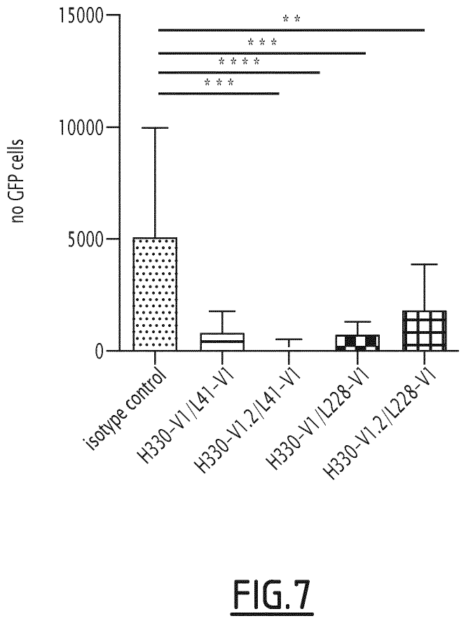

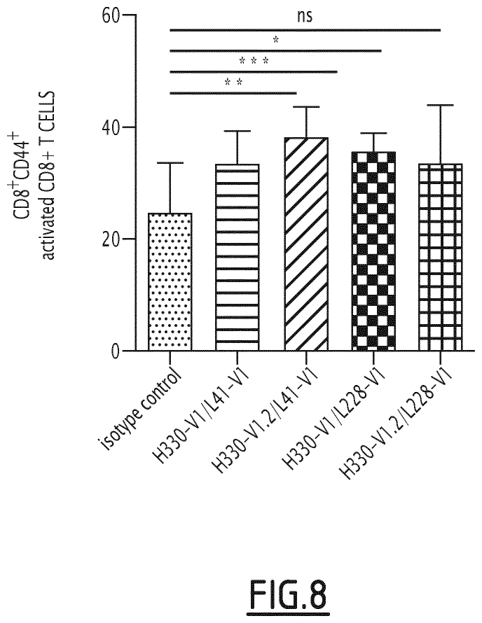

The results obtained are summarized in FIG. 3 for the chimeric 18B3 (considered as the reference mAb) as well as the 4 humanized lead variants, and a negative control. Statistical analysis (one-way ANOVA).

The results obtained showed that all the humanized variants have a statistically significant functionality against the target as compared to the negative control. This functionality is statistically fully comparable to the reference mAb (Chimeric 18B3). As a result, the in vitro functional test shows that the engineering of the selected humanized variants did not altered their in vitro functionality against their specific target. Although they are all very similar, ranking shows that variants with the Heavy chain H-330 gave the best results, variant H-330/L-41 being the most efficient

### Example 6: In Vivo Functional Bioassay

The purpose of this set of experiments is to demonstrate that co-administration of the 18B3 mAb together with PDAC specific tumor cells allows for limiting tumor growth (assessed by number of tumoral cells in the graft) and restoring CD8⁺ T cells activation pathway, as compared to control mice treated with isotype control IgG1 mAb.

The specific intent of this experiment is to show that the effects described above are proportional to the quantity of 18B3 administered.

PDAC tumor cells are obtained from dissociated pancreas of 2.5 months-old KIC mice and cultivated in-vitro.

KIC cells are embedded as plugs in a Matrigel 1:1 mix (Corning) and subcutaneously injected into the flanks of normal C57BL6 mice together with several increasing quantities of 18B3 mAb per mouse. The control consists in an irrelevant isotype control IgG1 mAb, administered at the highest 18B3 quantity to be assessed. Same mice population (n=8) is used for each quantity to be evaluated.

The mice are then monitored for 10 days and then sacrificed.

The tumor grafts are then weighed, measured and digested with collagenase buffer for obtaining single cell suspension and processed for staining prior to flow cytometry.

The quantity of tumoral cells in the graft and the proliferation and activation of CD8⁺ T cells are then assessed by FACS staining at 4° C. and analyzed with the FlowJo software.

The statistical significance of the parameters is assessed through Student t-test and One-Way Anova done by using GraphPad Prism Software.

The results show that: statistical differences are seen between humanized candidates and chimeric and between the various humanized candidates. Humanized variants bearing the Heavy Chain H-330 show better results overall. Humanized variant H-330/L-228 is the lead one, showing values equivalent or even better than Chimeric. This variant also exhibits the highest homogeneity (smallest dispersion—SEM) on all 3 parameters. The humanized variant H-330/L-228 shows the best profile.

### Example 7: Surface Plasmon Resonance (Biacore)—Affinity, Association and Dissociation Rates

The assays were conducted using Biacore T200 instrument. It was performed in 2 steps:
The first step, called suitability test, was aiming at determining the optimal conditions to run the assay: here, best sensor chip and running buffer conditions are assessed to defined best signal/noise ratio.
The second step is the run itself that was performed in triplicate to ensure reproducibility and robustness of the results.

For the first step, 2 sensors and various buffer conditions were assessed using the parental and Chimeric 18B3 mAbs as a reference. It was then defined that sensor Chip C1 was the most appropriate, and buffer containing 300 mM NaCl and 0.25 mg/mL BSA was reducing the non-specific binding and increased the signal.

For the run itself, the protocol was as following:
1. Reversible immobilization of test antibody via anti-mouse or anti-human IgG secondary antibody (covalently immobilized).
2. Interaction analysis of antigen with captured antibodies.
3. Regeneration: complete removal of antibody and antigen from secondary antibody surfaces S.

Additional details are given in the part Methods of measure.

Rate constants (kₐ, k_{d}) and equilibrium dissociation constants (K_{D}) of rhbIG-H3 interaction with six antibodies determined by SPR. Experimental data were fitted to a 1:1 binding model. Listed are mean values ±SD of n=3 independent experiments.

| | Kinetic fit (1:1 binding) | | | % of |
|---|---|---|---|---|
| Antibody | kₐ (M⁻¹s⁻¹) | K_{d} (s⁻¹) | K_{D} (M) | R_{max theor.} |
| Mouse | 5.72 (±0.35) E+05 | 2.34 (±0.07) E−04 | 4.11 (±0.32) E−10 | 301 (±10) % |
| monoclonal 18B3 | | | | |
| Chimeric huIgG1 | 1.32 (±0.03) E+06 | 2.47 (±0.11) E−04 | 1.87 (±0.06) E−10 | 327 (±5) % |
| k 18B3 | | | | |
| Humanized 18B3 | 1.50 (±0.03) E+06 | 7.26 (±0.27) E−04 | 4.82 (±0.10) E−10 | 333 (±16) % |
| A (H311-V1/L41- | | | | |
| V1) | | | | |
| Humanized 18B3 | 1.23 (±0.02) E+06 | 6.59 (±0.33) E−04 | 5.35 (±0.31) E−10 | 346 (±14) % |
| B (H330-V1/L41- | | | | |
| V1) | | | | |
| Humanized 18B3 | 1.45 (±0.05) E+06 | 7.07 (±0.15) E−04 | 4.90 (±0.14) E−10 | 299 (±1) % |
| D (H311-V1/L228- | | | | |
| V1) | | | | |
| Humanized 18B3 | 1.23 (±0.04) E+06 | 5.83 (±0.12) E−04 | 4.76 (±0.06) E−10 | 353 (±27) % |
| E (H330-V1/L228- | | | | |
| V1) | | | | |

The optimized setup of the SPR assay worked very well and provided very accurate and reproducible results.

All the mAbs have an affinity (KD) in the sub-nanomolar range.

There are overall few differences in ka, kd and KD of all samples:
The association rate (ka) of the mouse antibody m18B3 is slightly slower compared to all other samples,
The dissociation rate (kd) is surprisingly lower for the humanized (Hz) variants.
The overall KD value (affinity) is lowest for the chimeric antibody ch18B3 (0.2 nM) and very similar for the parental mouse and all humanized samples (0.4-0.5 nM).

### Example 8: Surface Plasmon Resonance—Affinity, Association and Dissociation Rates for the Antibodies Having Mutation C102S in H330

Affinity constant evaluation by SPR

Immobilization procedure

Running buffer (RB): HBS-EP+ composed of 10 mM HEPES, 0.15 M NaCl, 3 mM EDTA, 0.05% v/v

Surfactant P20, pH 7.4, Temperature: 25° C.

Anti-Human IgG (Fc) antibody is chemically grafted on a CM5 sensorchip using amine coupling according to the Cytivia 22064888AF notice. Briefly, surface is first activated by injection of a NHS-EDC mixture. Then, several injections (3 on the 10 available on the kit) of Anti-Human IgG (25 μg/ml in immobilization buffer) adjusting the contact time are performed. Finally, the surfaces are deactivated with ethanolamine 1M pH 8.5.

Immobilization of tested antibodies is performed at 5 μL/min sequentially on each flowcell until an immobilization level of 90-100 RU. Injection during 60 s of 20 μg/mL MAbs solutions (MAbs diluted with RB) led to an immobilization signal between 850 and 2500 RU. When high immobilization level was obtained, injection during 30 s of the regeneration solution (MgCl2 3M) was performed before a new injection with an adjusted concentration and contact time.

A negative control Ab is immobilized on sensor flowcell 1 (Fc 1) and positive control (chimeric 18B3 mAB) on Fc 2. 18B3 variants Abs are immobilized on Fc3 and Fc4:

| SCK-1 | Fc1 | Fc2 | Fc3 | Fc4 |
|---|---|---|---|---|
| mAb | X-HER3 | 18B3 chimeric | 18B3 H330- | 18B3 H330- |
| immobilized | (negative control) | (positive control) | V1.2/L41-V1 | V1.2/L41-V1 |
| Immobilization | 84 | 91 | 92 | 97 |
| level (RU) | | | | |
| SCK-2 | Fc1 | Fc2 | Fc3 | Fc4 |
| mAb | X-HER3 | 18B3 chimeric | 18B3 H330- | 18B3 H330- |
| immobilized | (negative control) | (positive control) | V1.2/L41-V1 | V1.2/L41-V1 |
| Immobilization | 99 | 102 | 95 | 101 |
| level (RU) | | | | |

### Single Cycle Kinetic (SCK) Assay

Running buffer (HBS-EP+): 10 mM HEPES, 0.15 M NaCl, 3 mM EDTA, 0.05% v/v Surfactant P20, pH 7.4, Temperature: 25° C. Flow rate: 30 μL/min

Injection of increasing concentrations (5; 10; 25; 50; 100 nM) of antigen (hβIG-H3) during 180 s is performed on each flowcell. Short dissociations are performed in RB between each antigen concentration. After injection of the highest antigen concentration (100 nM), a dissociation in RB is recorded during 3600 s.
Three similar cycles with five injections of RB are carried out before the cycle of Antigen in order to perform double subtraction procedure (Blank run).
Three similar cycles with five injection of RB and a dissociation time of 600 s are performed before the blank runs in order to stabilize the system (Startup run).
   Analysis with a 1:1 Interaction Model:

| | | | kA | kd | KD | Rmax | | | Chi² |
|---|---|---|---|---|---|---|---|---|---|
| | | mAb | (1/Ms) | (1/s) | (M) | (RU) | tc | kt | (RU²) |
| SCK1 | Fc2 | 18B3 chim | 2.06 · 10⁶ | 1.30 · 10⁻⁴ | 6.27 · 10⁻¹¹ | 81.02 | 1.37 · 10⁷ | 4.26 · 10⁷ | 1.31 |
| | Fc3 | 18B3 H330-V1.2/L41-V1 | 6.46 · 10⁶ | 7.98 · 10⁻⁴ | 1.24 · 10⁻¹⁰ | 55.53 | 6.75 · 10⁶ | 2.10 · 10⁷ | 2.29 |
| SCK2 | Fc4 | 18B3 H330-V1.2/L228-V1 | 4.66 · 10⁶ | 5.59 · 10⁻⁴ | 1.20 · 10⁻¹⁰ | 78.98 | 8.16 · 10⁶ | 2.54 · 10⁷ | 1.94 |
| | Fc2 | 18B3 chim | 1.87 · 10⁶ | 1.60 · 10⁻⁴ | 8.54 · 10⁻¹¹ | 86.84 | 9.92 · 10⁶ | 3.08 · 10⁷ | 0.863 |
| | Fc3 | 18B3 H330-V1.2/L41-V1 | 3.90 · 10⁶ | 6.59 · 10⁻⁴ | 1.69 · 10⁻¹⁰ | 70.08 | 6.77 · 10⁶ | 2.10 · 10⁷ | 2.25 |
| | Fc4 | 18B3 H330-V1.2/L228-V1 | 2.79 · 10⁶ | 4.78 · 10⁻⁴ | 1.71 · 10⁻¹⁰ | 82.69 | 6.9 · 10⁶ | 2.15 · 10⁷ | 1.81 |

### CONCLUSIONS

The 4 tested mAbs have a sub-nanomolar affinity (KD).
The affinity of humanized variants is very slightly lower than that of the chimeric version.
There is no significant difference between the 2 variants (H-330/L-228 and H-330/L-41), neither on the parental V1 versions, nor on the mutated V1.2 versions (C102->S102).
There is no significant difference, for each variant, between its parental version and its mutated version C102->S102. A very small difference can be observed and seems to be related to the experimental conditions because the tests were carried out in 2 series (i.e. using two SPR chips).

1. A humanized anti-βig-h3 monoclonal antibody or antigen-binding fragment thereof comprising a variable domain VH and a variable domain VL such as the antibody or antigen-binding fragment thereof binds specifically to an epitope of the βig-h3 protein, said epitope being as set forth as sequence SEQ ID NO: 16 or 30, which antibody or antigen-binding fragment thereof comprises:
(a) a variable domain VH having a sequence set forth as SEQ ID NO: 4 or 28,
(b) a variable domain VL which is a humanized variant of the murine 18B3 VL domain having a sequence set forth as SEQ ID NO: 18,
wherein this antibody or antigen-binding fragment displays a thermal stability in DSC (Tm Fab) which is of 79° C. or more, in particular between 79 or 80, and 83, 83.2 or 83.5° C.

2. The humanized anti-βig-h3 monoclonal antibody or an antigen-binding fragment thereof according to claim 1, which antibody or antigen-binding fragment thereof comprises:
a VH domain having a sequence set forth as SEQ ID NO: 4 or 28; and
a VL domain having a sequence set forth as SEQ ID NO: 10 or 13.

3. A humanized anti-βig-h3 monoclonal antibody or an antigen-binding fragment thereof comprising a variable domain VH and a variable domain VL such as the antibody or antigen-binding fragment thereof binds specifically to an epitope of the βig-h3 protein, said epitope being as set forth as sequence SEQ ID NO: 16 or 30, which antibody or antigen-binding fragment thereof comprises:
(a) a variable domain VH comprising:
a H-CDR1 having a sequence set forth as SEQ ID NO: 1;
a H-CDR2 having a sequence set forth as SEQ ID NO: 2;
a H-CDR3 having a sequence set forth as SEQ ID NO: 3 or 27;
(b) a variable domain VL comprising:
a L-CDR1 having a sequence set forth as SEQ ID NO: 7;
a L-CDR2 having a sequence set forth as SEQ ID NO: 8;
a L-CDR3 having a sequence set forth as SEQ ID NO: 9.

4. The humanized anti-βig-h3 monoclonal antibody or an antigen-binding fragment thereof according to claim 3, which antibody or antigen-binding fragment thereof:
binds to the βig-h3 protein with a K_{D} of about 5.8 E-10 M or less; and/or
binds to the βig-h3 protein with a K_{d} of about 6 E-04 s⁻¹ or more; and/or
has a stability in DSC (Tm Fab) of about 79° C. or more; and/or
has a productivity in transient expression in CHO cells of about 275 μg/ml.

5. The humanized anti-βig-h3 monoclonal antibody or antigen-binding fragment thereof according to claim 3, which antibody or antigen-binding fragment thereof comprises:
a VH domain having a sequence set forth as SEQ ID NO: 4 or 28;
a VL domain having a sequence set forth as SEQ ID NO: 10.

6. The humanized anti-βig-h3 monoclonal antibody according to claim 3, which antibody comprises:
a heavy chain comprising said variable domain and a constant domain CH, preferably a CH having a sequence set forth as SEQ ID NO: 14;
a light chain comprising said variable domain and a constant domain CL, preferably a CL having a sequence set forth as SEQ ID NO: 15.

7. The humanized anti-βig-h3 monoclonal antibody or antigen-binding fragment thereof according to claim 1, which antibody or antigen-binding fragment thereof and comprises:
(a) a variable domain VH comprising:
a H-CDR1 having a sequence set forth as SEQ ID NO: 1;
a H-CDR2 having a sequence set forth as SEQ ID NO: 2;
a H-CDR3 having a sequence set forth as SEQ ID NO: 3 or 27;
(b) a variable domain VL comprising:
a L-CDR1 having a sequence set forth as SEQ ID NO: 11;
a L-CDR2 having a sequence set forth as SEQ ID NO: 12;
a L-CDR3 having a sequence set forth as SEQ ID NO: 9.

8. The humanized anti-βig-h3 monoclonal antibody or an antigen-binding fragment thereof according to claim 7, which antibody or antigen-binding fragment thereof:
binds to the βig-h3 protein with a K_{D} of about 5 E-10 M or less; and/or
binds to the βig-h3 protein with a K_{d} of about 5 E-04 s⁻¹ or more; and/or
has a stability in DSC (Tm Fab) of 78° C. or more; and/or
has a productivity in transient expression in CHO cells of about 249 μg/ml.

9. The humanized anti-βig-h3 antibody or an antigen-binding fragment thereof according to claim 7, which antibody or antigen-binding fragment thereof comprises:
a VH domain having a sequence set forth as SEQ ID NO: 4 or 28; and
a VL domain having a sequence set forth as SEQ ID NO: 13.

10. The humanized anti-βig-h3 monoclonal antibody according to claim 7, which antibody comprises:
a heavy chain comprising said variable domain and a constant domain CH having a sequence set forth as SEQ ID NO: 14;
a light chain comprising said variable domain and a constant domain CL having a sequence set forth as SEQ ID NO: 15.

11. A humanized anti-βig-h3 monoclonal antibody or antigen-binding fragment thereof comprising a variable domain VH and a variable domain VL such as the antibody or antigen-binding fragment thereof binds specifically to an epitope of the βig-h3 protein, said epitope being as set forth as sequence SEQ ID NO: 16 or 30, which antibody or antigen-binding fragment thereof comprises:
a VH domain having a sequence set forth as SEQ ID NO: 6;
a VL domain having a sequence set forth as SEQ ID NO: 10 or 13.

12. A method of treatment of a solid cancer, comprising administering to a patient in need thereof a sufficient amount of a humanized anti-βig-h3 monoclonal antibody or an antigen-binding fragment thereof according to claim 1.

13. A method of treatment of a solid cancer, comprising administering to a patient in need thereof a sufficient amount of a humanized anti-βig-h3 monoclonal antibody or an antigen-binding fragment thereof according to claim 4.

14. The method of claim 13, wherein the cancer is a cancer in which the stromal protein βig-h3 is expressed.

15. The method according to claim 14, wherein the cancer is Pancreatic Ductal Adenocarcinoma (PDAC), lung cancer, head and neck cancer, colon cancer, bladder cancer, or melanoma.

16. (canceled)

17. The humanized anti-βig-h3 monoclonal antibody or an antigen-binding fragment thereof according to claim 1, which antibody or antigen-binding fragment thereof:
binds to the βig-h3 protein with a K_{D} of about 5.8 E-10 M or less; and/or
binds to the βig-h3 protein with a K_{d} of about 6 E-04 s⁻¹ or more; and/or
has a stability in DSC (Tm Fab) of about 79° C. or more; and/or
has a productivity in transient expression in CHO cells of about 275 μg/ml.

18. A method of treatment of a solid cancer, comprising administering to a patient in need thereof a sufficient amount of a humanized anti-βig-h3 monoclonal antibody or an antigen-binding fragment thereof according to claim 11.

19. The method of claim 13, wherein the cancer is Pancreatic Ductal Adenocarcinoma (PDAC), lung cancer, head and neck cancer, colon cancer, bladder cancer, or melanoma.

20. The method of claim 17, wherein the cancer is Pancreatic Ductal Adenocarcinoma (PDAC), lung cancer, head and neck cancer, colon cancer, bladder cancer, or melanoma.


SEQUENCE LISTING (32 sequences)

SEQ ID NO: 1 - protein, 5 aa, synthetic construct
  REGION 1..5: note = H-CDR1 H-330/311
DYYMY

SEQ ID NO: 2 - protein, 17 aa, synthetic construct
  REGION 1..17: note = H-CDR2 H-330
TISDGGIYKYYADSVKG

SEQ ID NO: 3 - protein, 11 aa, synthetic construct
  REGION 1..11: note = H-CDR3 H-330/311
GWDRYDSWFAC

SEQ ID NO: 4 - protein, 120 aa, synthetic construct
  REGION 1..120: note = VH H-330
QVQLVESGGGVVQPGGSLRLSCAASGFTFSDYYMYWVRQAPGKGLEWVATISDGGIYKYYADSVKGRFTISRDSSKNTLYLQMNSLRAEDTAVYYCVRGWDRYDSWFACWGQGTTVTVSS

SEQ ID NO: 5 - protein, 17 aa, synthetic construct
  REGION 1..17: note = H-CDR2 H-311
TISDGGIYIYYADSVKG

SEQ ID NO: 6 - protein, 120 aa, synthetic construct
  REGION 1..120: note = VH H-311
QVQLVESGGGLVKPGGSLRLSCAASGFTFSDYYMYWIRQAPGKGLEWVATISDGGIYIYYADSVKGRFTISRDSAKNSLYLQMNSLRAEDTAVYYCVRGWDRYDSWFACWGQGTTVTVSS

SEQ ID NO: 7 - protein, 17 aa, synthetic construct
  REGION 1..17: note = L-CDR1 L-41
KSSQSLLYSSNQKNYLA

SEQ ID NO: 8 - protein, 7 aa, synthetic construct
  REGION 1..7: note = L-CDR2 L-41
WASTRES

SEQ ID NO: 9 - protein, 9 aa, synthetic construct
  REGION 1..9: note = L-CDR3 L-41/228
QQYYRYPYT

SEQ ID NO: 10 - protein, 113 aa, synthetic construct
  REGION 1..113: note = VL L-41
DIVMTQSPDSLAVSLGERATMNCKSSQSLLYSSNQKNYLAWYQQKPGQPPKLLIYWASTRESGVPDRFSGSGSGTDFTLTISSLQAEDVAVYYCQQYYRYPYTFGQGTKLEIK

SEQ ID NO: 11 - protein, 17 aa, synthetic construct
  REGION 1..17: note = L-CDR1 L-228
RSSQSLLYSSNQKNYLA

SEQ ID NO: 12 - protein, 7 aa, synthetic construct
  REGION 1..7: note = L-CDR2 L-228
WGSTRES

SEQ ID NO: 13 - protein, 113 aa, synthetic construct
  REGION 1..113: note = VL L-228
DIVMTQSPLSLPVTPGEPASMSCRSSQSLLYSSNQKNYLAWYLQKPGQSPQLLIYWGSTRESGVPDRFSGSGSGTDFTLKISRVEAEDVGVYYCQQYYRYPYTFGQGTKLEIK

SEQ ID NO: 14 - protein, 330 aa, Homo sapiens [sequence omitted]

SEQ ID NO: 15 - protein, 107 aa, Homo sapiens [sequence omitted]

SEQ ID NO: 16 - protein, 10 aa, Homo sapiens
ALPPRERSRL

SEQ ID NO: 17 - protein, 120 aa, synthetic construct
  REGION 1..120: note = VH 18B3
EVQLVESGGGLVKPGGSLKLSCAASGFTFSDYYMYWVRQTPEKRLEWVATISDGGIYTYYPDSVKGRFTISRDSAKNNLYLQMTSLKSDDTAMYYCVRGWDRYDSWFACWGQGTLVTVSA

SEQ ID NO: 18 - protein, 113 aa, synthetic construct
  REGION 1..113: note = VL 18B3
DIVMSQSPSSLVVSAGEKVTMTCKSSQSLLYSSNQKNYLAWYRQKPGQSPKLLIYWASTRESGVPDRFTGSGSGTDFTLTISSVKAEDLAVYYCQQYYRYPYTFGGGTKLEIK

SEQ ID NO: 19 - DNA, 360 bp, synthetic construct
  source 1..360: mol_type = other DNA
  misc_feature 1..360: note = coding sequence VH 18B3
gaagtgcagctggtggaaagcggcggcggcctggtgaaaccgggcggcagcctgaaactgagctgcgcggcgagcggctttacctttagcgattattatatgtattgggtgcgccagaccccggaaaaacgcctggaatgggtggcgaccattagcgatggcggcatttatacctattatccggatagcgtgaaaggccgctttaccattagccgcgatagcgcgaaaaacaacctgtatctgcagatgaccagcctgaaaagcgatgataccgcgatgtattattgcgtgcgcggctgggatcgctatgatagctggtttgcgtgctggggccagggcaccctggtgaccgtgagcgcg

SEQ ID NO: 20 - DNA, 339 bp, synthetic construct
  source 1..339: mol_type = other DNA
  misc_feature 1..339: note = coding sequence VL 18B3
gatattgtgatgagccagagcccgagcagcctggtggtgagcgcgggcgaaaaagtgaccatgacctgcaaaagcagccagagcctgctgtatagcagcaaccagaaaaactatctggcgtggtatcgccagaaaccgggccagagcccgaaactgctgatttattgggcgagcacccgcgaaagcggcgtgccggatcgctttaccggcagcggcagcggcaccgattttaccctgaccattagcagcgtgaaagcggaagatctggcggtgtattattgccagcagtattatcgctatccgtatacctttggcggcggcaccaaactggaaattaaa

SEQ ID NO: 21 - DNA, 360 bp, synthetic construct
  source 1..360: mol_type = other DNA
  misc_feature 1..360: note = coding sequence VH H-330
caggtgcagctggtggaaagcggcggcggcgtggtgcagccgggcggcagcctgcgcctgagctgcgcggcgagcggctttacctttagcgattattatatgtattgggtgcgccaggcgccgggcaaaggcctggaatgggtggcgaccattagcgatggcggcatttataaatattatgcggatagcgtgaaaggccgctttaccattagccgcgatagcagcaaaaacaccctgtatctgcagatgaacagcctgcgcgcggaagataccgcggtgtattattgcgtgcgcggctgggatcgctatgatagctggtttgcgtgctggggccagggcaccaccgtgaccgtgagcagc

SEQ ID NO: 22 - DNA, 360 bp, synthetic construct
  source 1..360: mol_type = other DNA
  misc_feature 1..360: note = coding sequence VH H-311
caggtgcagctggtggaaagcggcggcggcctggtgaaaccgggcggcagcctgcgcctgagctgcgcggcgagcggctttacctttagcgattattatatgtattggattcgccaggcgccgggcaaaggcctggaatgggtggcgaccattagcgatggcggcatttatatttattatgcggatagcgtgaaaggccgctttaccattagccgcgatagcgcgaaaaacagcctgtatctgcagatgaacagcctgcgcgcggaagataccgcggtgtattattgcgtgcgcggctgggatcgctatgatagctggtttgcgtgctggggccagggcaccaccgtgaccgtgagcagc

SEQ ID NO: 23 - DNA, 339 bp, synthetic construct
  source 1..339: mol_type = other DNA
  misc_feature 1..339: note = coding sequence VL L-41
gatattgtgatgacccagagcccggatagcctggcggtgagcctgggcgaacgcgcgaccatgaactgcaaaagcagccagagcctgctgtatagcagcaaccagaaaaactatctggcgtggtatcagcagaaaccgggccagccgccgaaactgctgatttattgggcgagcacccgcgaaagcggcgtgccggatcgctttagcggcagcggcagcggcaccgattttaccctgaccattagcagcctgcaggcggaagatgtggcggtgtattattgccagcagtattatcgctatccgtatacctttggccagggcaccaaactggaaattaaa

SEQ ID NO: 24 - DNA, 339 bp, synthetic construct
  source 1..339: mol_type = other DNA
  misc_feature 1..339: note = coding sequence VL L-228
gatattgtgatgacccagagcccgctgagcctgccggtgaccccgggcgaaccggcgagcatgagctgccgcagcagccagagcctgctgtatagcagcaaccagaaaaactatctggcgtggtatctgcagaaaccgggccagagcccgcagctgctgatttattggggcagcacccgcgaaagcggcgtgccggatcgctttagcggcagcggcagcggcaccgattttaccctgaaaattagccgcgtggaagcggaagatgtgggcgtgtattattgccagcagtattatcgctatccgtatacctttggccagggcaccaaactggaaattaaa

SEQ ID NO: 25 - DNA, 990 bp, Homo sapiens
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 26 - DNA, 321 bp, Homo sapiens
  source 1..321: mol_type = other DNA
aggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 27 - protein, 11 aa, synthetic construct
GWDRYDSWFAS

SEQ ID NO: 28 - protein, 120 aa, synthetic construct
QVQLVESGGGVVQPGGSLRLSCAASGFTFSDYYMYWVRQAPGKGLEWVATISDGGIYKYYADSVKGRFTISRDSSKNTLYLQMNSLRAEDTAVYYCVRGWDRYDSWFASWGQGTTVTVSS

SEQ ID NO: 29 - DNA, 360 bp, synthetic construct
  source 1..360: mol_type = other DNA
caggtgcagctggtggaaagcggcggcggcgtggtgcagccgggcggcagcctgcgcctgagctgcgcggcgagcggctttacctttagcgattattatatgtattgggtgcgccaggcgccgggcaaaggcctggaatgggtggcgaccattagcgatggcggcatttataaatattatgcggatagcgtgaaaggccgctttaccattagccgcgatagcagcaaaaacaccctgtatctgcagatgaacagcctgcgcgcggaagataccgcggtgtattattgcgtgcgcggctgggatcgctatgatagctggtttgcgagctggggccagggcaccaccgtgaccgtgagcagc

SEQ ID NO: 30 - protein, 8 aa, Homo sapiens
LPPRERSR

SEQ ID NO: 31 - protein, 113 aa, synthetic construct
EIVMTQSPATLSVSPGERATMSCRASQSLLYSSNQKNYLAWYQQKPGQAPRLLIYWASTRETGIPARFSGSGSGTEFTLTISSLQSEDFAVYYCQQYYRYPYTFGQGTKLEIK

SEQ ID NO: 32 - protein, 120 aa, synthetic construct
QVQLVQSGAEVKKPGSSVKVSCKASGFTFSDYYMYWVRQAPGQGLEWVGTISDGGIYAYYAQKVQGRFTITRDSSTSTLYLELSSLRSEDTAVYYCVRGWDRYDSWFACWGQGTTVTVSS